(19) United States
(12) Patent Application Publication
(10) Pub. No.: US 2026/0257256 A1
(43) Pub. Date: Sep. 3, 2026

(54) DEGRADATION-RESISTANT BIOMASS POLYMER COMPOSITES FOR CARBON REMOVAL

(71) Applicant: Graphyte, Inc., Memphis, TN (US)

(72) Inventors: Hannah MURNEN, Havertown, PA (US); Saptarshi DATTA, Memphis, TN (US)

(21) Appl. No.: 19/550,757

(22) Filed: Feb. 26, 2026

Related U.S. Application Data
(60) Provisional application No. 63/765,197, filed on Feb. 28, 2025.

(51) Int. Cl. B09B 3/20; A61L 2/04; A61L 2/10; B09B 3/32; B09B 3/35; B09B 101/85; B65B 9/08; B65B 55/14; B65B 63/02

(52) U.S. Cl. CPC B09B 3/20; A61L 2/04; A61L 2/10; B09B 3/32; B09B 3/35; B65B 9/08; B65B 55/14; B65B 63/02; B09B 2101/85

(57) ABSTRACT

Systems and methods for processing, storing, and/or monitoring biomass are generally described. The systems and methods include in different described embodiments one or more of the following process step or capabilities for sequestering carbon from biomass: comminuting the biomass; sterilizing or reducing bioburden or the ability to sustain biological activity of the comminuted biomass, for example by heated drying, consolidating biomass; encapsulating consolidated biomass; and quantifying a carbon content of the biomass. Certain systems and methods further include the capability for detecting leakage or degradation of processed biomass sequestered in a biomass storage facility.

### CROSS-REFERENCE TO RELATED APPLICATION

The present application claims the benefit of priority to U.S. Provisional Application No. 63/765,197, filed on Feb. 28, 2025, which is incorporated by reference herein in its entirety.

### TECHNICAL FIELD

Systems and methods for carbon sequestration via processing, storing, and/or monitoring biomass are generally described.

### BACKGROUND

Atmospheric levels of gaseous compounds comprising carbon (e.g., CO₂, CH₄) have been increasing for hundreds of years, with the increasing atmospheric levels of these gases being correlated to global climate change. Extensive research and governmental policy have been directed to managing the increasing levels of atmospheric carbon, including using cleaner technologies (e.g., electric-powered vehicles instead of petroleum-powered vehicles) and adopting policies to promote greener alternatives (e.g., renewable energy incentives, carbon sequestration tax credits). Despite the push to decrease emissions, atmospheric carbon levels continue to increase. Accordingly, improved systems and methods to remove atmospheric carbon are needed.

### SUMMARY

Systems and methods for carbon sequestration via processing, storing, and/or monitoring biomass are generally described. The subject matter of the present disclosure involves, in some cases, interrelated products, alternative solutions to a particular problem, and/or a plurality of different uses of one or more systems and/or articles.

In one aspect, a method for sequestering carbon is described. In some embodiments, the method comprises receiving biomass. In some embodiments, the method comprises processing the biomass for sequestration. In certain embodiments, processing comprises comminuting the biomass; sterilizing the comminuted biomass; consolidating the biomass to form a plurality of consolidated biomass units; encapsulating the consolidated biomass units; and quantifying a carbon content of the biomass.

In another aspect, a method for sequestering carbon is described. In some embodiments, the method comprises receiving biomass. In some embodiments, the method comprises processing the biomass for sequestration. In certain embodiments, processing comprises sterilizing the biomass; encapsulating the sterilized biomass; and quantifying a carbon content of the encapsulated biomass.

In another aspect, a method for sequestering carbon is described. In some embodiments, the method comprises receiving biomass. In some embodiments, the method comprises processing the biomass for sequestration. In certain embodiments, processing comprises sterilizing the biomass; consolidating the biomass to form a plurality of consolidated biomass units; encapsulating the consolidated biomass units; and quantifying a carbon content of the biomass.

In another aspect, a method for sequestering carbon is described. In some embodiments, the method comprises receiving biomass. In some embodiments, the method comprises processing the biomass for sequestration. In certain embodiments, processing comprises consolidating the biomass. In certain embodiments, processing comprises consolidating the biomass to form a plurality of consolidated biomass units; encapsulating the consolidated biomass units sufficiently to prevent microbial activity and decay of encapsulated biomass for at least 100 years when the biomass is stored in darkness under standard atmospheric conditions; and quantifying a carbon content of the biomass. In certain cases, the method involves encapsulating the consolidated biomass units sufficiently to prevent microbial activity and decay of encapsulated biomass for at least 100 years when the biomass is stored in conditions where exposure to light is possible, such as in an above-ground warehouse or similar.

In another aspect, a method for sequestering carbon is described. In some embodiments, the method comprises receiving processed biomass. In certain embodiments, the processed biomass has been consolidated and encapsulated. In some embodiments, the method comprises monitoring at least one property of the processed biomass or an area where the processed biomass is stored to determine the stability and/or sterility of the processed biomass. In certain embodiments, the monitoring comprises sampling gas from a hermetically sealed area in which the processed biomass is stored through a first outlet; and opening a vent to the hermetically sealed area when sampling gas through the first inlet to maintain a pressure within the hermetically sealed area.

In another aspect, a method for sequestering carbon is described. In some embodiments, the method comprises receiving biomass. In some embodiments, the method comprises processing the biomass for sequestration. In certain embodiments, processing comprises comminuting the biomass. In certain embodiments, processing comprises sterilizing the comminuted biomass. In certain embodiments, processing comprises consolidating the biomass to form a plurality of consolidated biomass units. In certain embodiments, processing comprises encapsulating the consolidated biomass units. In certain embodiments, processing comprises quantifying a carbon content of the biomass.

In another aspect, an article is described. In some embodiments, the article comprises biomass. In certain embodiments, the biomass is substantially resistant to microbial growth. In certain embodiments, the biomass has a sterility assurance level of 10-1 or less. In some embodiments, the biomass of the article is substantially free of non-biomass material. In some embodiments, the article comprises one or more layers surrounding the biomass. In certain embodiments, the one or more layers are substantially impervious to oxygen, water, and/or carbon dioxide.

In one aspect, a biomass-containing article is described. The article comprises biomass; one or more barrier layers surrounding the biomass, and a tracer that is not biomass or a degradation product of biomass that is detectable by a sensor to indicate a disruption of the one or more barrier layers and/or a change in the mass of the biomass resulting from leakage or degradation.

In another aspect, a method for monitoring for degradation of units of biomass and/or a leak from units of biomass in a biomass storage system containing stored biomass is described. The method comprises detecting at least one component of a tracer in the event of a release of the tracer within or from within the biomass storage system; and determining a location of leakage or degradation of at least one leaking or degrading unit of biomass within the biomass storage system.

In another embodiment the method involves monitoring for degradation of biomass and/or a leak in a biomass in a storage system containing a plurality of stored biomass units. Such method comprises detecting at least one component of a tracer released from a compromised biomass unit of the plurality of stored biomass units in the event of a release of the tracer from the biomass unit within the biomass storage system; and determining a location of the compromised biomass unit within the biomass storage system.

In another embodiment the method involves providing a discrete unit of a processed biomass material; and encapsulating the unit of the processed biomass material with an encapsulating layer comprising a polymer that is not a thermoplastic polymer such that the unit of the processed biomass material is hermetically sealed therein.

In another embodiment the method includes providing a discrete unit of a processed biomass material and encapsulating the unit of the processed biomass material with an encapsulating layer such that the unit of the processed biomass material is hermetically sealed within the encapsulating layer, wherein the encapsulating layer comprises: a thermosetting synthetic polymer and/or a natural, non-synthetically produced polymer or resin.

In another aspect, materials are described. In some embodiments, a material comprising a discrete unit of a processed biomass material and an encapsulating layer surrounding the unit of the processed biomass material that comprises a polymer that is not a thermoplastic polymer, wherein the unit of the processed biomass material is hermetically sealed within the encapsulating layer.

In another embodiment, the material comprising a discrete unit of a processed biomass material; and an encapsulating layer surrounding the unit of the processed biomass material, wherein the encapsulating layer comprises a thermosetting synthetic polymer and/or a natural, non-synthetically produced polymer or resin.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates a multi-step method for sequestering carbon, according to some embodiments;

FIG. 2A shows a schematic illustration of comminution of unprocessed biomass, according to some embodiments;

FIG. 2B shows a schematic illustration of sterilization of comminuted biomass, according to some embodiments;

FIG. 2C shows a schematic illustration of consolidation of sterilized biomass, according to some embodiments;

FIG. 2D shows schematic illustrations of consolidated biomass encapsulated in a layer, according to some embodiments;

FIG. 2E shows a schematic illustration of a cross-sectional view of the exemplary encapsulated biomass of FIG. 2D, according to some embodiments;

FIG. 2F shows a schematic illustration of consolidated biomass encapsulated in a first layer and a second layer, according to some embodiments;

FIG. 2G shows a schematic illustration of a cross-sectional view of the exemplary encapsulated biomass of FIG. 2F, according to some embodiments;

FIG. 2H shows a schematic illustration of a pallet comprising a plurality of blocks of encapsulated biomass;

FIG. 2I shows a schematic illustration of a sequestration site comprising a plurality of blocks of encapsulated biomass;

FIG. 3 shows a schematic illustration of a system for monitoring one or more properties of biomass at a sequestration site; and

FIG. 4 is a plot of % CO₂ produced by biomass under simulated sequestered conditions, where the tested biomass was sterilized by dehydration with heated air according to the conditions of Example 3.

FIG. 5 shows an exemplary method for coextrudating biomass with a polymer and densifying the coextruded biomass.

FIG. 6 shows an exemplary method for testing the hydrophobicity of the densified biomass of FIG. 5.

FIG. 7 shows an exemplary method for testing degradation of the densified biomass of FIG. 5.

FIG. 8 shows cumulative CO₂ evolution (y-axis) over time in days (x-axis) for composite biomass and undried biomass. The biomass was stored in a chamber with humidity close to 100%. The composite material evolved less CO₂, indicating that it was more resistant to decomposition.

FIG. 9 shows shows cumulative CO₂ evolution (y-axis) over time in days (x-axis) for composite pellets and dry, densified (carbon casted) biomass. The composite pellets were exposed to 100% humidity, while the dry, densified biomass was exposed to 35-40% humidity. The composite pellets and dry, densified biomass evolved similar levels of CO₂ over time with the composite biomass's evolution being only slightly higher.

### DETAILED DESCRIPTION

Utilizing biomass for carbon removal requires that the carbon within the biomass is prevented from decomposing and being released as carbon dioxide or methane. This is currently done through processing (i.e., drying) the biomass and storing it in conditions that prevent decay (i.e., in dry or anoxic conditions). However, if it were possible to process the biomass itself such that it was inherently recalcitrant to decomposition, that would reduce the need for protection during storage. In that case, if water were to get into the storage chamber, the biomass itself would repel it and therefore prevent decomposition from occurring.

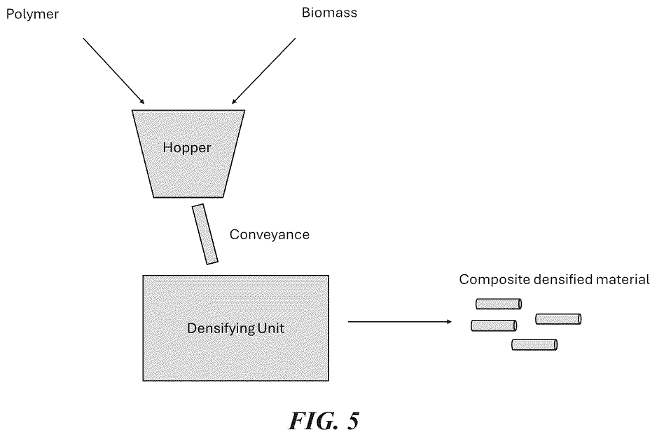

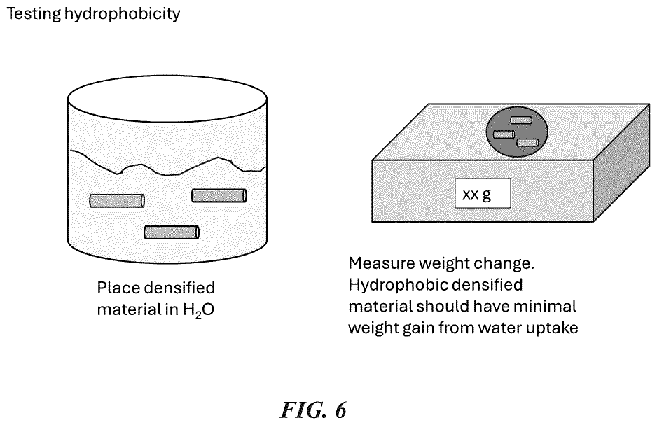

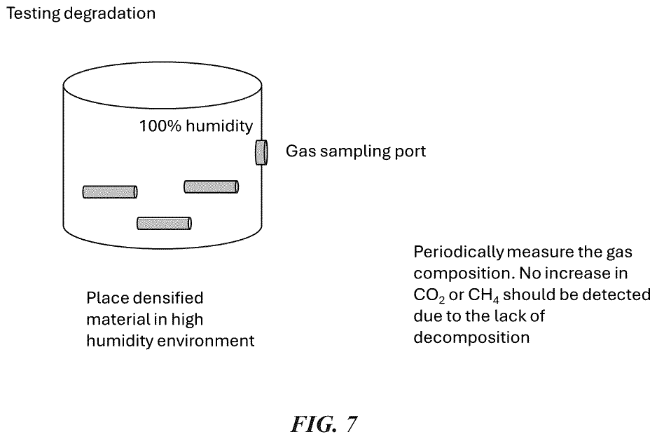

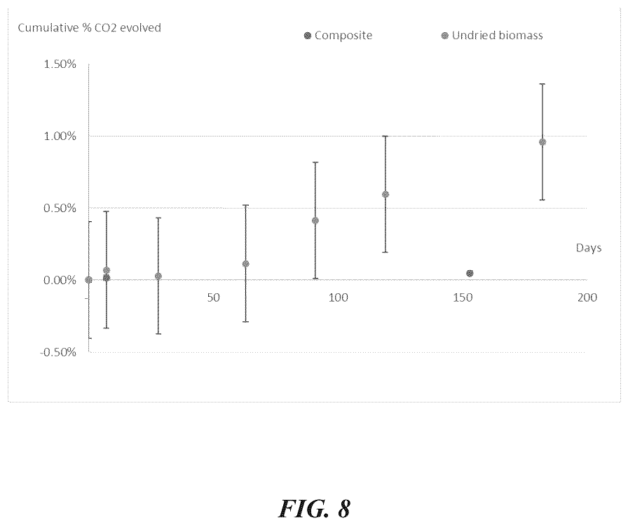

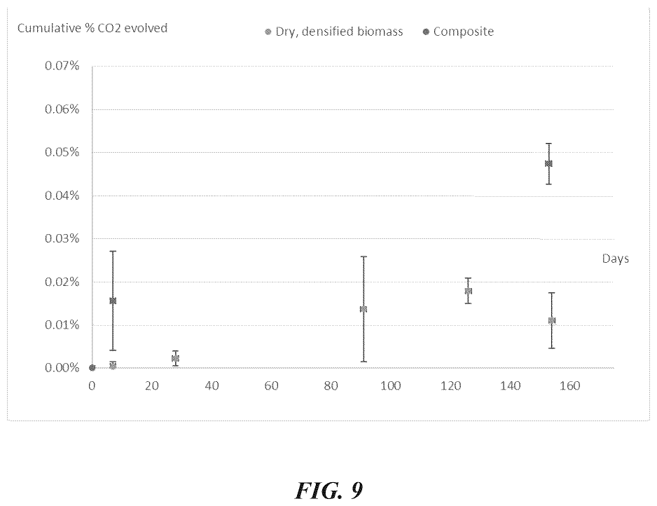

This disclosure is to create a hydrophobic composite material that is recalcitrant to decomposition through the simultaneous densification of biomass with a polymer stream. Ideally this would be done with waste polymer that would otherwise need disposal. For example, coextruding polymer with biomass into a pellet creates a composite pellet that can be water-repellant. This water-repellant behavior can make the composite pellet recalcitrant to decomposition, an ideal set of characteristics for carbon removal. In embodiments, provided herein is biomass, which is coextruded with a polymer, which is hydrophobic. In embodiments, biomass, which is coextruded with a polymer, which is hydrophobic, is not wrapped in an encapsulating layer. In embodiments, biomass, which is coextruded with a polymer, which is hydrophobic, is sequestered. In embodiments, the biomass, which is coextruded with a polymer, which is hydrophobic, is stored in a storage facility. In embodiments, the biomass that is coextruded with a polymer is densified as shown in FIG. 5. In embodiments, densified biomass has a density from about 300 kg/m³ to about 1500 kg/m³. In embodiments, the biomass that is coextruded with a polymer and/or densified is resistant to degradation. Degradation may be tested as in FIG. 7. In embodiments, the biomass that is coextruded with a polymer and/or densified is hydrophobic. The biomass's hydrophobicity may be measured according to FIG. 6. In embodiments, the storage facility is a landfill. In embodiments, the polymer is any polymer described herein. In embodiments, the polymer is any polymer found in U.S. Publication No. 2016/0145519 or U.S. Publication No. 2009/0235577, each which is incorporated by reference herein in its entirety for all purposes.

Systems and methods related to processing, storing, and/or monitoring biomass are generally described. Some aspects of the present system and/or methods are related to sequestering carbon. In some cases, unprocessed biomass is received and processed to form processed biomass. For example, in some cases, biomass may be comminuted, dried/dehydrated, sterilized, consolidated, and/or encapsulated in one or more layers (e.g., one or more layers comprising a polymeric material). According to some embodiments, processed biomass may be stored to sequester carbon contained within the biomass. Still other aspects are directed to monitoring the processed biomass (e.g., to evaluate the stability of the sequestered carbon as a function of time).

One strategy for reducing the level of carbon in the atmosphere involves capturing carbon dioxide (CO₂) from the air and storing or using the captured CO₂ such that it cannot reenter the atmosphere. Some methods of capturing atmospheric CO₂ are energy intensive—for example, direct air capture, which utilizes large mechanical systems and solid adsorbents or liquid solvents to capture CO₂, may require 5-7 GJ to remove a ton of CO₂ from the atmosphere. In contrast, plants naturally remove CO₂ from the air through sunlight-powered photosynthesis. Accordingly, some aspects of the present disclosure relate to capturing carbon in the form of biomass (e.g., plant-derived biomass).

Although there are existing biomass-based approaches to carbon sequestration, these existing approaches may have limited effectiveness over the long term (e.g., at least 100 years). For example, some existing biomass-based approaches to carbon sequestration involve burying unprocessed (or minimally processed) biomass in landfills or subterranean formations. However, decomposition of biomass releases CO₂ and/or CH₄, and decomposition of unprocessed (or minimally processed) biomass in uncontrolled environments may result in highly variable levels of CO₂ and/or CH₄ being produced over time depending on biomass type (e.g., biomass with higher carbohydrate content may degrade more quickly than biomass with higher lignin content) and/or environmental conditions (e.g., greater exposure to microbes, water, and/or oxygen may result in more rapid decomposition). In addition, in some cases, unprocessed (or minimally processed) biomass (e.g., wood logs) may be challenging to transport and/or store efficiently.

Some aspects of the disclosure are directed to systems and methods that overcome key challenges associated with existing biomass-based carbon sequestration approaches. For example, some aspects of the disclosure relate to processing biomass to reduce or eliminate biomass decomposition and thereby reduce or eliminate production of carbon-containing gases (e.g., CO₂, CH₄). Certain aspects of systems and methods described herein relate to reduction or elimination of decomposition and/or microbial activity via sterilization of biomass (e.g., to reduce or eliminate microbes present or sustainability of microbial activity in the biomass), consolidation of the biomass, and encapsulation of the consolidated biomass in one or more layers (e.g., one or more layers comprising a polymeric material). In some cases, such aspects may ensure that biomass decomposition terminates (following sterilization) and does not restart and/or cannot be sustained under storage conditions (since the one or more encapsulation layers are preferably impervious to water, oxygen, water vapor, and/or microbes). Certain aspects of systems and methods described herein further relate to storage of processed biomass in a sequestration site (e.g., a landfill, a subterranean location). According to some embodiments, consolidating the biomass (e.g., into pellets, extruded cylindrical logs, briquettes and/or blocks) before storing the biomass may enable more compact, stable, and structurally sound storage of the biomass. In some cases, such aspects may ensure that any carbon-containing gases from biomass decomposition are not released into the atmosphere.

Such systems and methods may advantageously capture atmospheric carbon and minimize or prevent release of carbon-containing gases into the atmosphere (e.g., by reducing or eliminating microbial decomposition of biomass and ensuring that such biomass decomposition does not recommence), thereby decreasing atmospheric carbon levels over the long term (e.g., at least 100 years, at least 500 years, at least 1000 years, at least 1500 years, at least 2000 years, at least 2500 years, at least 5000 years, at least 10,000 years). In certain cases, such systems and methods may achieve such reduced atmospheric carbon levels without adding large amounts of additives (e.g., salts), which may be costly and/or environmentally deleterious, to the biomass.

Certain aspects also relate to the high-accuracy quantification of carbon content within the biomass. Such aspects may allow for precise recording of the amount of carbon captured and stored within a particular sequestration site. Certain aspects also relate to monitoring of various aspects of the stored biomass and/or sequestration site. In some cases, such aspects may advantageously allow the stability of sequestered carbon to be monitored over the long term.

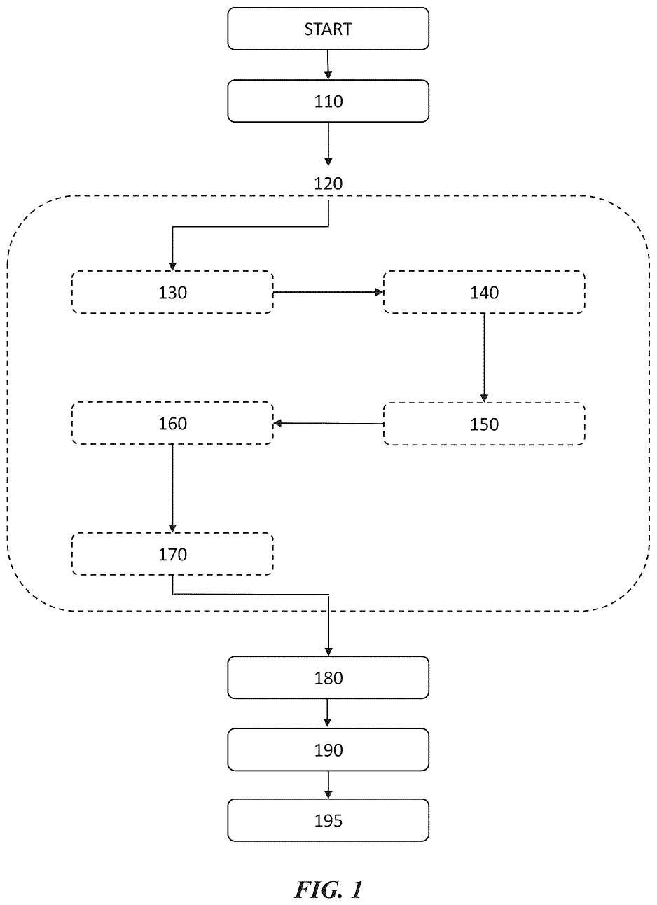

A schematic diagram of an exemplary embodiment for processing, storing, and monitoring biomass for carbon sequestration is shown in the non-limiting example of FIG. 1. Here, biomass may be received from a source 110 (e.g., a farm, a forest, an agricultural processing facility, a lumber processing facility, etc.). The biomass may be processed 120. Processing the biomass may comprise any of a variety of steps, for example, comminuting the biomass 130, sterilizing the biomass 140, consolidating the biomass 150, encapsulating the biomass 160, and/or quantifying the carbon content of the biomass 170. The processed biomass may then be transported 180, stored 190, and/or monitored 195.

The foregoing example is but one embodiment of the methods described herein. The processing steps are optional, but in some cases, it may be advantageous to include some or all of the steps. Moreover, the arrangement of the steps shown in FIG. 1 is not the only order contemplated. As another non-limiting example, in some cases, biomass may be encapsulated and then sterilized. Other combinations and/or configurations of steps shown in FIG. 1 are possible, some of which are described elsewhere herein. Each step shown in FIG. 1 is described in further detail below.

In some embodiments, biomass is first obtained (e.g., received). Any of a variety of types and/or sources of biomass may be suitable for later processing, storing, and/or monitoring steps. In some embodiments, the biomass is plant-derived biomass. In some embodiments, the plant-derived biomass may be residue or waste resulting from conversion of a precursor biomass feedstock into a biofuel or other product of a chemical transformation. According to some such embodiments, the plant-derived biomass considered waste of such a conversion process is the sequestered biomass for the carbon sequestration processes described herein. In another embodiment, a plant-derived biomass may have one portion thereof that is converted to a biofuel and a residual portion that is sequestered. For example, in one embodiment, the biomass is a corn plant, wherein a first portion of the corn plant (e.g., the kernels) is converted into a biofuel such as ethanol, a second portion of the corn plant (e.g., corn stover) is sequestered via the carbon sequestration processes described herein. Such an arrangement may be desirable, according to some embodiments, as the carbon sequestration process associated with the second portion of the biomass may lower the overall carbon intensity associated with the use of the biofuel converted from the first portion of the biomass. Carbon intensity is known to those of ordinary skill in the art and is generally considered the metric tons of carbon dioxide equivalents per megajoule of energy produced from the biomass source used to produce the biofuel.

In certain embodiments, the plant-derived biomass comprises waste from agricultural harvesting and/or processing. Non-limiting examples of suitable waste from agricultural harvesting and processing include palm oil waste, sugarcane bagasse, rice husks, soybean hulls, coconut shell husks, rice straw, wheat straw, and corn stover. In certain embodiments, the plant-derived biomass comprises waste from lumber harvesting and/or processing. Non-limiting examples of suitable waste from lumber harvesting and/or processing include logs, lumber residue, bark, sawdust, wood chips, boles, and branches. In certain embodiments, the plant-derived biomass comprises grasses (e.g., fast-growing grasses). Non-limiting examples of grasses include miscanthus and switchgrass. Other suitable types of plant-derived biomass include, but are not limited to, yard scraps (e.g., lawn clippings, branches, leaves, mowed grass), and seaweed. In some embodiments, using plant-derived biomass comprising organic waste from agricultural or lumber harvesting and/or processing may minimize costs due to some sources of organic waste being of limited, if any, benefit for other applications. In some embodiments, the biomass is animal-derived biomass (e.g., animal waste). Non-limiting examples of animal waste include poultry litter and feedlot effluent. In some embodiments, the biomass comprises the organic fraction of municipal solid waste. In some embodiments, the biomass comprises food waste (e.g., food discarded by grocery stores and/or restaurants, food past its expiration date, etc.). In some cases, the obtained biomass may be solid biomass and/or liquid biomass. In accordance with some embodiments, it may be advantageous to use solid biomass, as opposed to liquid biomass, because the liquid biomass may require more energy to process than solid biomass. For example, processing liquid biomass may require an initial substantial and energy-intensive dehydration step before further processing can occur (and/or may require more extensive dehydration than solid biomass).

Obtaining biomass may comprise any of a variety of suitable methods. The biomass may be received from any of a variety of sources. Non-limiting examples of suitable sources include farms, forests, agricultural processing facilities (e.g., agricultural mills, palm oil processing facilities, sugar refineries, rice mills), lumber processing facilities (e.g., lumber mills, paper mills), forestry companies, municipal governments, grocery stores, restaurants, biofuel producers, and food processing facilities. In some cases, obtaining biomass comprises purchasing and/or receiving biomass from a vendor (e.g., a farm, forest, an agricultural processing facility, a lumber processing facility, a forestry company, a grocery store, a restaurant, a food processing facility) and/or an institution collecting compost waste (e.g., a municipal government). In some cases, organic waste may be manually collected. According to some embodiments, biomass may be intentionally grown for carbon sequestration purposes (e.g., a fast-growing crop, such as miscanthus and/or switchgrass) and subsequently harvested according to any known harvesting technique (e.g., using a combine harvester). Other methods for obtaining biomass are possible.

Some aspects of the present disclosure are related to processing the biomass. Processing of the biomass, in some cases, may comprise any one or combination of the following steps, not limited to a particular order. That is, in some cases, the processing of the biomass may occur in the order as recited below. In other cases, some and/or all of the steps for processing the biomass may occur in a different order. Additionally, none, some, and/or all of the steps for processing the biomass may occur, in addition to storing and/or monitoring the biomass.

According to some embodiments, processing the biomass comprises comminuting the biomass. In some cases, comminuting the biomass advantageously facilitates further processing (e.g., sterilizing, consolidating, encapsulating) of the biomass. For example, comminuting the biomass may increase the surface area to volume ratio of the biomass, allowing further processing steps (e.g., drying and/or sterilizing) to be performed more effectively and/or efficiently. In addition, in some cases, comminuting may result in a flowable solid, which may facilitate consolidation of the biomass into a particular size and/or shape.

In some cases, the biomass to be comminuted may be unprocessed. In other cases, the biomass may have been processed via one or more other processing steps disclosed herein before being comminuted. According to some embodiments, the biomass may not be comminuted. For example, in some cases, the biomass (e.g., sawdust) may be a suitable size upon being received and may not be comminuted.

In some embodiments, comminuting the biomass comprises grinding, shredding, pounding, chopping, milling, and/or cutting the biomass. Comminuting the biomass may be performed using any suitable device. Non-limiting examples of suitable devices include grinders, shredders, hammer mills (e.g., as typically used during wood pelletization), chippers, flakers, refiners, and ball mills. Non-limiting examples of suitable shredders include Weima WL 4, WL 6, and WL 8 shredders. Those of ordinary skill and art will recognize various methods and devices for comminuting biomass. According to some embodiments, it may be advantageous to align certain biomass sources to facilitate comminuting the biomass. For example, when the biomass comprises straw (e.g., wheat straw, rice straw), it may be advantageous to align a long axis of the straw with a direction of movement into the device in which the straw is to be comminuted. This may facilitate comminution of biomass such as straw that has large aspect ratios (e.g., greater than or equal to 2:1, greater than or equal to 5:1, greater than or equal to 10:1; greater than or equal to 20:1, greater than or equal to 50:1, greater than or equal to 100:1, and/or less than or equal to 500:1, or less than or equal to 1,000:1.

In some embodiments, comminuting the biomass results in biomass particles having a relatively small average size. For example, in accordance with some embodiments, articles of biomass having a first average largest dimension may be comminuted to particles having a second average largest dimension, wherein the second average largest dimension is less than the first average largest dimension.

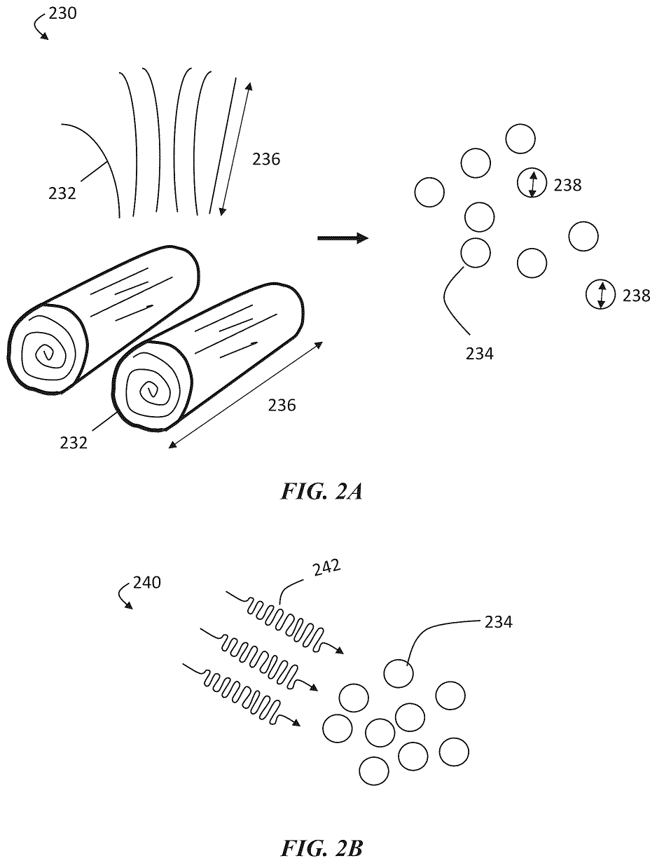

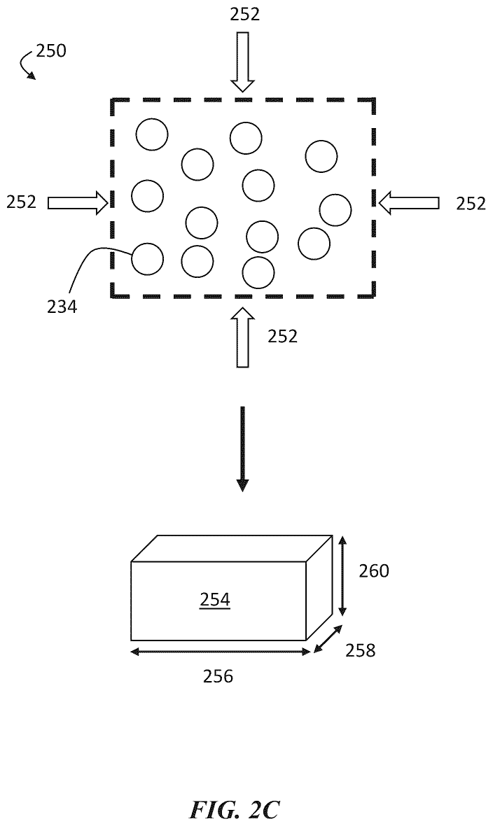

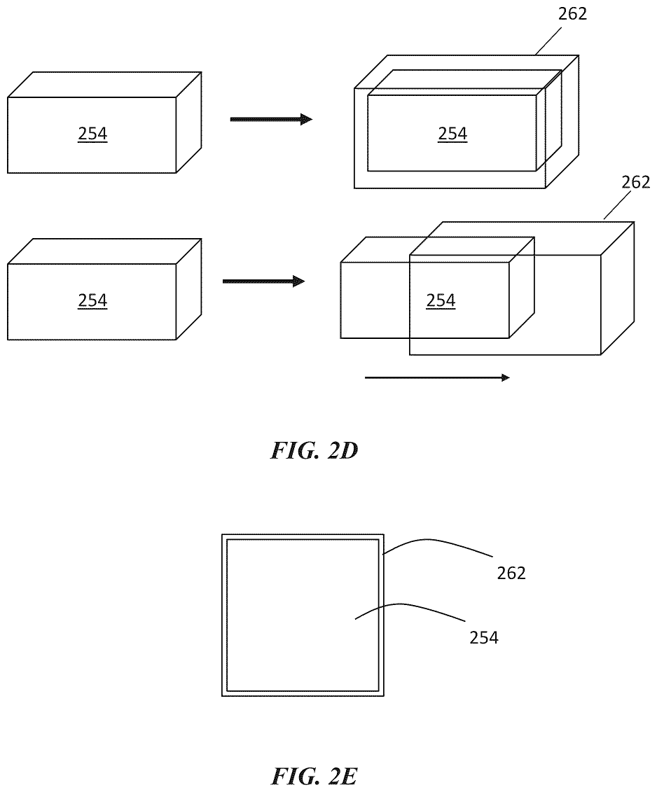

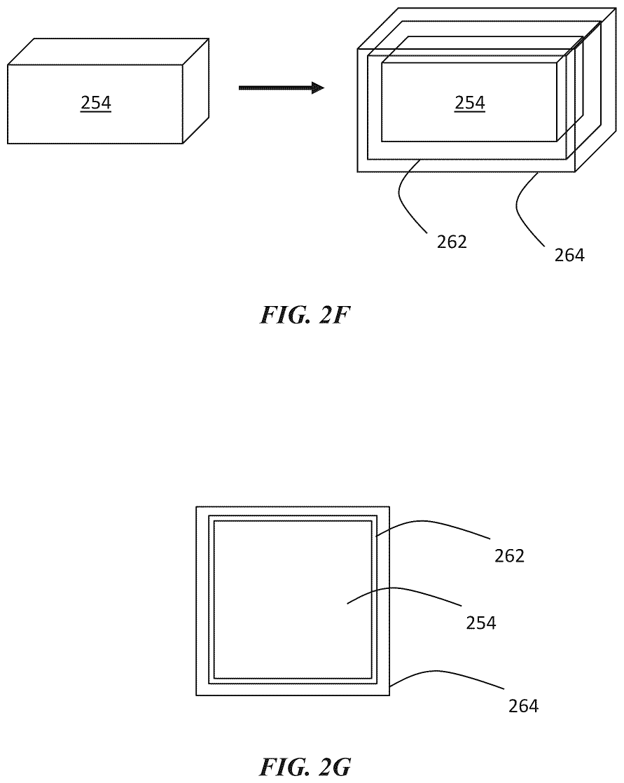

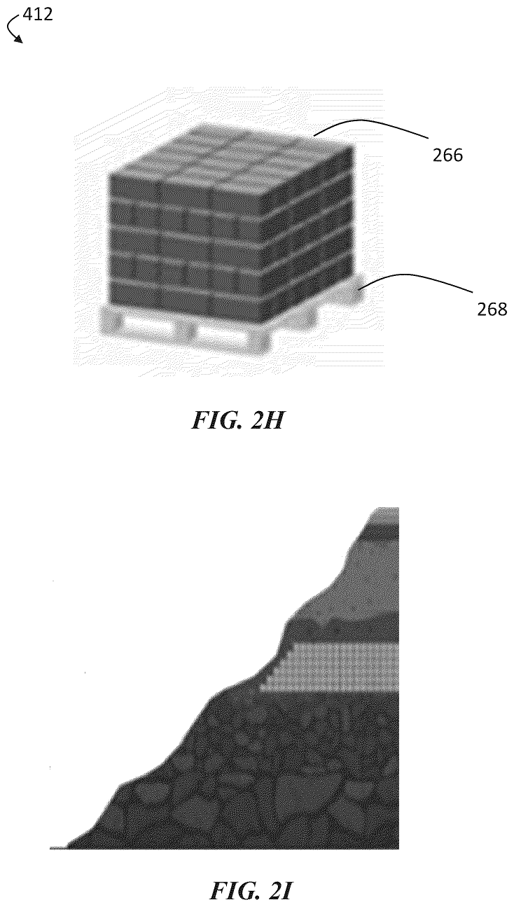

FIG. 2A shows a schematic illustration of a non-limiting example of comminuting biomass 230. In FIG. 2A, biomass 232, such as grass or lumber, may be received and comminuted 233 (e.g., ground, shredded, etc.) into a plurality of relatively uniform particles 234. As shown in FIG. 2A, non-comminuted articles of biomass 232 have an average largest dimension 236 that is larger than an average largest dimension 238 of particles 234.

In some cases, the articles of the non-comminuted biomass may have any of a variety of average largest dimensions. In some cases, the articles of the non-comminuted biomass have a first average largest dimension that is greater than or equal to 5 cm, greater than or equal to 10 cm, greater than or equal to 20 cm, greater than or equal to 50 cm, greater than or equal to 1 m, greater than or equal to 2 m, greater than or equal to 3 m, greater than or equal to 5 m, greater than or equal to 10 m, greater than or equal to 15 m, greater than or equal to 20 m, or greater than or equal to 25 m. In some cases, the first average largest dimension of articles of the biomass is less than or equal to 25 m, less than or equal to 20 m, less than or equal to 15 m, less than or equal to 10 m, less than or equal to 5 m, less than or equal to 3 m, less than or equal to 2 m, less than or equal to 1 m, less than or equal to 50 cm, less than or equal to 20 cm, less than or equal to 10 cm, or less than or equal to 5 cm. Combinations of the foregoing ranges are possible (e.g., greater than or equal to 5 cm and less than or equal to 25 m). Other ranges are also possible.

According to some embodiments, comminution may result in particles of the biomass having a second average largest dimension. In some cases, the second average largest dimension may be greater than or equal to 1 micron, greater than or equal to 10 microns, greater than or equal to 50 microns, greater than or equal to 100 microns, greater than or equal to 500 microns, greater than or equal to 1 mm, greater than or equal to 2 mm, greater than or equal to 3 mm, greater than or equal to 5 mm, greater than or equal to 1 cm, greater than or equal to 2 cm, greater than or equal to 3 cm, greater than or equal to 4 cm, or greater than or equal to 5 cm. In some embodiments, the second average largest dimension may be less than or equal to 5 cm, less than or equal to 4 cm, less than or equal to 3 cm, less than or equal to 2 cm, less than or equal to 1 cm, less than or equal to 5 mm, less than or equal to 3 mm, less than or equal to 2 mm, less than or equal to 1 mm, less than or equal to 500 microns, less than or equal to 100 microns, less than or equal to 50 microns, less than or equal to 10 microns, or less than or equal to 1 micron. Combinations of the foregoing ranges are possible (e.g., greater than or equal to 1 micron and less than or equal to 5 cm). Other ranges are also possible.

According to some embodiments, the comminution may result in particles having a relatively uniform size. Particles having a relatively uniform size, in some embodiments, may behave like a flowable solid, which may facilitate further processing and/or consolidation of the biomass into a particular size and/or shape. In some embodiments, a size of an individual particle of comminuted biomass may vary by no more than 100%, no more than 90%, no more than 80%, no more than 70%, no more than 60%, no more than 50%, no more than 40%, no more than 30%, no more than 20%, no more than 10%, no more than 5%, or no more than 1% of the second average largest dimension of a the comminuted particles.

In some embodiments, comminuting the biomass may be performed in an at least partially enclosed facility. In certain embodiments, comminuting the biomass may be performed in a wholly enclosed (e.g., indoor) facility. In some embodiments, comminuting the biomass may be performed in an outdoor environment.

According to some embodiments, processing the biomass comprises sterilizing the biomass. In some embodiments, the sterilizing step occurs after a comminuting step and before a consolidating step. In some embodiments, the sterilizing step occurs after a consolidating step and before an encapsulating step. In some cases, the comminuted biomass may be sterilized. In other cases, unprocessed biomass may be sterilized.

Sterilizing is to take its normal meaning in the art and will be understood by those of ordinary skill in the art. Generally, sterilizing indicates the at least partial removal, deactivation, and/or elimination of life, for example, microbes (e.g., methanogens, CO₂-producing microbes), within the biomass, thereby minimizing and/or preventing decomposition of the biomass by the microbes. In some cases, the biomass may be encapsulated in one or more layers (e.g., one or more layers comprising a polymeric material) as described elsewhere herein before and/or after being sterilized, and the sterilization of the biomass may sufficiently reduce the number of microbes present such that decomposition of the biomass is slowed and/or halted within the one or more layers (e.g., one or more layers comprising a polymeric material).

In some embodiments, sterilizing the biomass may comprise any of a variety of suitable methods, which will be understood by those of ordinary skill in the art. In some cases, sterilizing the biomass comprises heating the biomass. In certain cases, heating the biomass comprises exposing the biomass to dry heat and/or wet heat (e.g., steam) using any suitable heating device. Non-limiting examples of suitable heating devices include ovens, autoclaves, water bath devices, water cascade devices, heat exchangers, dryers (e.g., rotary drum dryers, fluidized bed dryers, rolling bed dryers, microwave dryers), convection furnaces, radiant heaters, and solar receivers/dryers/heaters.

In some embodiments, sterilizing the biomass comprises heating the biomass at a sterilization temperature for a sterilization time. In some cases, the sterilization temperature is at least 65° C., at least 70° C., at least 80° C., at least 90° C., at least 100° C., at least 120° C., at least 150° C., at least 170° C., at least 200° C., at least 300° C., at least 400° C., at least 500° C., at least 600° C., at least 700° C., at least 800° C., or at least 850° C. In some cases, the sterilization temperature is in a range from 65° C. to 80° C., 65° C. to 90° C., 65° C. to 100° C., 65° C. to 120° C., 65° C. to 150° C., 65° C. to 200° C., 65° C. to 500° C., 65° C. to 850° C., 70° C. to 80° C., 70° C. to 90° C., 70° C. to 100° C., 70° C. to 120° C., 70° C. to 150° C., 70° C. to 200° C., 70° C. to 500° C., 70° C. to 850° C., 80° C. to 90° C., 80° C. to 100° C., 80° C. to 120° C., 80° C. to 150° C., 80° C. to 200° C., 80° C. to 500° C., 80° C. to 850° C., 90° C. to 100° C., 90° C. to 120° C., 90° C. to 150° C., 90° C. to 200° C., 90° C. to 500° C., 90° C. to 850° C., 100° C. to 120° C., 100° C. to 150° C., 150° C. to 180° C., 100° C. to 200° C., 100° C. to 500° C., 100° C. to 850° C., 120° C. to 150° C., 120° C. to 200° C., 120° C. to 500° C., 120° C. to 850° C., 150° C. to 200° C., 150° C. to 500° C., 150° C. to 850° C., 200° C. to 500° C., 200° C. to 850° C., or 500° C. to 850° C. In some cases, the sterilization temperature is about 170° C. In some embodiments, the sterilization time is at least 5 seconds, at least 15 seconds, at least 30 seconds, at least 1 minute, at least 3 minutes, at least 5 minutes, at least 10 minutes, at least 15 minutes, at least 30 minutes, at least 45 minutes, at least 60 minutes, at least 90 minutes, at least 120 minutes, at least 150 minutes, or at least 180 minutes. In some embodiments, the sterilization time is 180 minutes or less, 150 minutes or less, 120 minutes or less, 90 minutes or less, 60 minutes or less, 45 minutes or less, 30 minutes or less, 15 minutes or less, 10 minutes or less, 5 minutes or less, 3 minutes or less, 1 minute or less, 30 seconds or less, 15 seconds or less, or 5 seconds or less. In certain embodiments, the sterilization time is in a range from 5 seconds to 15 seconds, 5 seconds to 30 seconds, 5 seconds to 1 minute, 5 seconds to 3 minutes, 5 seconds to 5 minutes, 5 seconds to 10 minutes, 5 seconds to 15 minutes, 5 seconds to 30 minutes, 5 seconds to 45 minutes, 5 seconds to 60 minutes, 5 seconds to 90 minutes, 5 seconds to 120 minutes, 5 seconds to 150 minutes, 5 seconds to 180 minutes, 30 seconds to 1 minute, 30 seconds to 3 minutes, 30 seconds to 5 minutes, 30 seconds to 10 minutes, 30 seconds to 15 minutes, 30 seconds to 30 minutes, 30 seconds to 45 minutes, 30 seconds to 60 minutes, 30 seconds to 90 minutes, 30 seconds to 120 minutes, 30 seconds to 150 minutes, 30 seconds to 180 minutes, 1 minute to 5 minutes, 1 minute to 10 minutes, 1 minute to 15 minutes, 1 minute to 30 minutes, 1 minute to 45 minutes, 1 minute to 60 minutes, 1 minute to 90 minutes, 1 minute to 120 minutes, 1 minute to 150 minutes, 1 minute to 180 minutes, 5 minutes to 10 minutes, 5 minutes to 15 minutes, 5 minutes to 30 minutes, 5 minutes to 45 minutes, 5 minutes to 60 minutes, 5 minutes to 90 minutes, 5 minutes to 120 minutes, 5 minutes to 150 minutes, 5 minutes to 180 minutes, 10 minutes to 30 minutes, 10 minutes to 45 minutes, 10 minutes to 60 minutes, 10 minutes to 90 minutes, 10 minutes to 120 minutes, 10 minutes to 150 minutes, 10 minutes to 180 minutes, 30 minutes to 60 minutes, 30 minutes to 90 minutes, 30 minutes to 120 minutes, 30 minutes to 150 minutes, 30 minutes to 180 minutes, 60 minutes to 90 minutes, 60 minutes to 120 minutes, 60 minutes to 150 minutes, 60 minutes to 180 minutes, 90 minutes to 120 minutes, 90 minutes to 150 minutes, 90 minutes to 180 minutes, 120 minutes to 150 minutes, 120 minutes to 180 minutes, or 150 minutes to 180 minutes. In some embodiments, the sterilization time is about 20 minutes. In a specific embodiment, the sterilization temperature is about 170° C. and the sterilization time is about 20 minutes.

In some cases, sterilizing the biomass comprises exposing the biomass to electromagnetic radiation (e.g., microwave, x-ray, gamma ray, and/or ultraviolet (UV) radiation). In some cases, sterilizing the biomass comprises exposing the biomass to one or more chemical disinfectants (e.g., sodium hypochlorite, ethylene oxide, ozone, chlorine gas, vaporized hydrogen peroxide, formaldehyde vapor). In some cases, sterilizing the biomass comprises neutralizing methanogens and/or CO₂-producing microbes. In some cases, sterilizing the biomass comprises vacuum sealing the biomass. Still other methods for sterilizing the biomass are possible. FIG. 2B shows an exemplary embodiment of sterilizing biomass 240. In FIG. 2B, comminuted biomass 234 is exposed to UV radiation 242, which sterilizes the comminuted biomass 234.

In some embodiments, sterilizing the biomass comprises performing a single step described herein (e.g., heating/drying the biomass, exposing the biomass to electromagnetic radiation or a chemical disinfectant, etc.). In some embodiments, sterilizing the biomass comprises performing two or more steps described herein. As an illustrative example, sterilizing the biomass may comprise a first step of exposing the biomass to UV radiation (e.g., radiation having a wavelength in a range from 100 nm to 400 nm), a second step of dehydrating the biomass, and a third step of heating the biomass.

In some embodiments, sterilized biomass has a sufficiently high assurance of sterility to prevent subsequent microbial growth under anticipated time periods and conditions of sequestration. The sterility assurance level (“SAL”) of a product provides a measure of the probability that the product will remain nonsterile after undergoing a sterilization process. As an illustrative example, an SAL of 10⁻³ means that there is a 1 in 1,000 chance of a viable microorganism being present in a sterilized product. In some cases, the SAL of the sterilized biomass is less than 10⁰, 10⁻¹ or less, 10⁻² or less, 10⁻³ or less, 10⁻⁴ or less, 10⁻⁵ or less, or 10⁻⁶ or less. As used herein, a SAL of “10ⁿ or less” encompasses a SAL of 10ⁿ, 10ⁿ⁻¹, 10ⁿ⁻², 10ⁿ⁻³, etc.

In some embodiments, a sterilization process described herein may achieve a desired log reduction in the population of a targeted microorganism (sometimes referred to as a “challenge microorganism”). In certain embodiments, the challenge organism is a gram-positive bacterium, a methanogen, and/or a CO₂-producing microbe. In some embodiments, the sterilized biomass has at least a 1 log reduction, at least a 2 log reduction, at least a 3 log reduction, at least a 4 log reduction, at least a 5 log reduction, or at least a 6 log reduction in the population of a challenge microorganism relative to the unsterilized biomass. In some embodiments, the sterilized biomass has a log reduction in the population of a challenge microorganism relative to the unsterilized biomass in a range from 1 log reduction to 2 log reduction, 1 log reduction to 3 log reduction, 1 log reduction to 4 log reduction, 1 log reduction to 5 log reduction, 1 log reduction to 6 log reduction, 2 log reduction to 3 log reduction, 2 log reduction to 4 log reduction, 2 log reduction to 5 log reduction, 2 log reduction to 6 log reduction, 3 log reduction to 4 log reduction 3 log reduction to 5 log reduction, 3 log reduction to 6 log reduction, 4 log reduction to 5 log reduction, 4 log reduction to 6 log reduction, or 5 log reduction to 6 log reduction.

In some embodiments, sterilizing the biomass may be performed in an at least partially enclosed facility. In certain embodiments, sterilizing the biomass may be performed in a wholly enclosed (e.g., indoor) facility. In some cases, performing the sterilizing step in a wholly enclosed (e.g., indoor) facility may advantageously reduce or eliminate contamination during the sterilization process.

According to some embodiments, processing the biomass comprises dehydrating the biomass. In certain cases, a single step (e.g., heating or microwaving the biomass) may achieve both dehydration and sterilization of the biomass. In some embodiments, two or more steps (e.g., performed simultaneously or sequentially) may be used to dehydrate and sterilize the biomass. In some embodiments, dehydrating the biomass comprises heating, microwaving, filtering, centrifuging, mechanically dewatering, and/or chemically desiccating the biomass. According to some embodiments, a rotary drum dryer may be used to heat and dehydrate the biomass.

Dehydrating the biomass may comprise decreasing the initial moisture wt. % (i.e., an initial water content) of the biomass to a final moisture wt. % (i.e., a final water content) of the biomass. In some embodiments, the biomass may be at least partially dehydrated using a heated air dryer, such as a rotary drum dryer. For instance, within such a heated air dryer, the biomass may be exposed to heated air at any of a variety of suitable temperatures (e.g., at temperature of between 150° C. to 200° C., etc. as described elsewhere herein) for any of a variety of temperatures (e.g., for 5 minutes to 60 minutes, etc. as described elsewhere herein) to dehydrate the biomass, In some cases, it may be desirable to partially dehydrate the biomass without completely dehydrating the biomass (e.g., wherein the biomass has 0 moisture wt. %) to aid in the consolidation process, as described elsewhere herein. At least partially dehydrating the biomass, in some embodiments, involves decreasing a water activity of the biomass to a level to effectively sterilize the biomass to a degree of sterility sufficient to prevent degradation during subsequent processing and sequestration. In some embodiments, partial dehydration (e.g., to a final moisture content is greater than or equal to 1 wt. %, greater than or equal to 2 wt. %, greater than or equal to 3 wt. %, greater than or equal to 4 wt. %, etc.) may be desirable as the dehydration step may be more less energy intensive and/or more efficient and/or render the biomass more amenable to consolidation or other subsequent processing steps, e.g., when compared to full or complete dehydration where a water content is 0 wt. % or close thereto, such as in conventional methods of sterilizing biomass via drying. In some such embodiments, the remaining water content in the biomass is chosen to sufficiently sterilize the biomass for the purposes of stability for sequestration using the methods described herein, e.g., in certain embodiments, the final moisture content is chosen to be insufficient to support microbial growth such that dehydrating and sterilizing the biomass occurs in a single step while decreasing the energy necessary to dehydrate the biomass when compared to typical conventional drying methods. According to some embodiments, the biomass may be dehydrated until the final moisture content is greater than or equal to 1 wt. %, greater than or equal to 2 wt. %, greater than or equal to 4 wt. %, greater than or equal to 6 wt. %, greater than or equal to 8 wt. %, greater than or equal to 10 wt. %, greater than or equal to 12 wt. %, greater than or equal to 14 wt. %, greater than or equal to 16 wt. %, greater than or equal to 18 wt. %, greater than or equal to 20 wt. %, greater than or equal to 22 wt. %, greater than or equal to 24 wt. %, greater than or equal to 26 wt. %, greater than or equal to 28 wt. %, or greater than or equal to 30 wt. % of the biomass. In some cases, the final moisture content of the biomass may be less than or equal to 30 wt. %, less than or equal to 28 wt. %, less than or equal to 26 wt. %, less than or equal to 24 wt. %, less than or equal to 22 wt. %, less than or equal to 20 wt. %, less than or equal to 18 wt. %, less than or equal to 16 wt. %, less than or equal to 14 wt. %, less than or equal to 12 wt. %, less than or equal to 10 wt. %, less than or equal to 8 wt. %, less than or equal to 6 wt. %, less than or equal to 4 wt. %, less than or equal to 2 wt. %, or less than or equal to 1 wt. % of the biomass. Combinations of the foregoing ranges are possible (e.g., greater than or equal to 1 wt. % and less than or equal to 30 wt. %, greater than or equal to 6 wt. % and less than or equal to 14 wt. %, greater than or equal to 10 wt. % and less than or equal to 12 wt. %, greater than or equal to 4 wt. % and less than or equal to 30 wt. %, greater than or equal to 4 wt. %, and less than or equal to 14 wt. %, greater than or equal to 4 wt. % and less than or equal to 12 wt. %, greater than or equal to 4 wt. % and less than or equal to 10 wt. %). Other ranges are also possible.

According to some embodiments, a first portion of the biomass may be sterilized (e.g., via heating and dehydrating the biomass), whereas a second portion of the biomass may not need to be sterilized. According to some embodiments, as described above, sterilizing the biomass may comprise dehydrating the biomass to lower a moisture content present in the biomass to prevent microbial growth. Accordingly, if a portion of the biomass naturally has a moisture content insufficient to sustain microbial growth, it may be unnecessary to perform a sterilization step on such biomass. Thus, in some embodiments, a first portion of biomass may be dehydrated, while a second portion of the biomass may not undergo a dehydration step. For example, in some embodiments, the biomass may be plant-derived biomass as described elsewhere herein, where a portion of the biomass is rice hull and/or sawdust that is sufficiently dry such that it does not contain enough moisture to facilitate microbial growth. In such embodiments, the biomass comprising rice hull and/or sawdust may not need to be dehydrated or subjected to a sterilization step. Other biomass sources that may have a sufficiently low moisture content to prevent microbial growth may need not be sterilized are also treatable using methods and systems described herein.

In some embodiments, processing the biomass comprises consolidating the biomass. Consolidating the biomass, in some cases, may make the biomass easier to process (e.g., encapsulate), stack, transport, handle, store, and/or monitor. For instance, in some embodiments, consolidating the biomass may include forming a plurality of consolidated biomass units, each of which may facilitate further processing and/or manipulation. In certain embodiments, consolidating the biomass may advantageously result in units of consolidated biomass that can withstand relatively high compressive and/or shear loads and/or can resist rupture of one or more encapsulation layers. In some cases, the biomass being consolidated may be comminuted biomass and/or sterilized biomass. In some embodiments, consolidating the biomass may comprise applying pressure to at least a portion of the biomass such that the consolidated biomass has a higher density than the unconsolidated biomass (i.e., densifying the biomass). In certain embodiments wherein the biomass comprises lignin, sufficient pressure may be applied to cause at least a portion of lignin of the biomass to crosslink.

Consolidating the biomass may be performed using any suitable device. Non-limiting examples of suitable devices include extruders, presses (e.g., stamping presses, hydraulic presses, screw presses), briquetting machines, pelletizers, and cuber machines. Those of ordinary skill and art will recognize various methods and devices for consolidating biomass.

FIG. 2C shows a schematic illustration of a non-limiting example of consolidating biomass 250. In FIG. 2C, pressure 252 is applied to comminuted biomass 234 from multiple directions to form consolidated biomass 254. In FIG. 2C, consolidated biomass 258 is a rectangular block having a first dimension 256, a second dimension 258, and a third dimension 260, which may be any of a variety of sizes.

In some embodiments, consolidating the biomass may comprise mixing the biomass with one or more additives. In certain embodiments, the one or more additives may be added to the biomass (e.g., before consolidation) to enhance the structural properties and/or prevent decomposition of the consolidated biomass material. For example, in some embodiments, the one or more additives comprise one or more cross-linking agents or other adhesives. In some cases, the one or more cross-linking agents comprise one or more monomers and/or oligomers that may crosslink within the biomass. According to some embodiments, the biomass may be heated and/or exposed to radiation (e.g., UV radiation) after being consolidated, which may induce cross-linking of the one or more cross-linking agents and thereby increase the structural integrity of the consolidated biomass. In some embodiments, the one or more additives comprise a desiccant (e.g., alumina, silica gel, and/or CaCl₂) that dehydrates the biomass. In some embodiments, the one or more additives comprise one or more anti-microbial agents (e.g., antibacterial compounds). In some embodiments, the one or more additives comprise a tracer (e.g., isotopically-labeled molecule, tracer gas). In some such cases, the tracer may be useful for monitoring decomposition and/or other compromised condition of the biomass as described elsewhere herein. In some embodiments, mixtures of different tracers may be used to provide greater resolution to assist in determining a location within a biomass storage facility of a decomposing, leaking, or otherwise compromised unit of stored biomass. For example, by providing more unique tracer “signatures” characterizing different biomass containing units or storage locations for the same total number of unique tracers, e.g., the use of tracers A and B individually in biomass stored units provide the ability to discriminate between leakage from each of the two units, but if a mixture of A+B is included, a third point of discrimination can be obtained. Similarly, different ratios of A to B in an A+B mixture can provide additional ability to discriminate. Addition of even more unique tracers (i.e., three or more) in different combinations and/or ratios can lead to even further detectable markers for leak location and/or origin determination.

In some embodiments, any additive added to the biomass may be added in a relatively small amount. In certain embodiments, any additive added to the biomass may be present in an amount less than or equal to 5 wt. %, less than or equal to 4 wt. %, less than or equal to 3 wt. %, less than or equal to 2 wt. %, less than or equal to 1.5 wt. %, less than or equal to 1 wt. %, or less than or equal to 0.05 wt. %. In certain embodiments, any additive added to the biomass may be present in an amount in a range from 0.05 wt. % to 1 wt. %, 0.05 wt. % to 1.5 wt. %, 0.05 wt. % to 2 wt. %, 0.05 wt. % to 3 wt. %, 0.05 wt. % to 4 wt. %, 0.05 wt. % to 5 wt. %, 1 wt. % to 2 wt. %, 1 wt. % to 3 wt. %, 1 wt. % to 4 wt. %, 1 wt. % to 5 wt. %, 2 wt. % to 3 wt. %, 2 wt. % to 4 wt. %, 2 wt. % to 5 wt. %, 3 wt. % to 4 wt. %, 3 wt. % to 5 wt. %, or 4 wt. % to 5 wt. %.

According to some embodiments, applying a pressure to consolidate the biomass may comprise applying any suitable pressure. In some cases, the pressure may be applied anisotropically to the biomass. In other cases, the pressure may be applied isotropically to the biomass to uniformly consolidate the biomass. In certain embodiments, the pressure may be applied from one direction (e.g., from a top direction, from a bottom direction). In certain embodiments, the pressure may be applied from two or more directions (e.g., from top and bottom directions, from top, bottom, and one to four side directions).

In some embodiments, applying a pressure to consolidate the biomass comprises applying a pressure greater than or equal to 1 MPa, greater than or equal to 2 MPa, greater than or equal to 3 MPa, greater than or equal to 4 MPa, greater than or equal to 5 MPa, greater than or equal to 6 MPa, greater than or equal to 7 MPa, greater than or equal to 8 MPa, greater than or equal to 9 MPa, greater than or equal to 10 MPa, greater than or equal to 20 MPa, greater than or equal to 30 MPa, greater than or equal to 40 MPa, greater than or equal to 50 MPa, greater than or equal to 80 MPa, greater than or equal to 100 MPa, greater than or equal to 150 MPa, greater than or equal to 200 MPa, greater than or equal to 250 MPa, greater than or equal to 300 MPa, greater than or equal to 350 MPa, or greater than or equal to 400 MPa. In some embodiments, applying a pressure to consolidate the biomass comprises applying a pressure less than or equal to 400 MPa, less than or equal to 350 MPa, less than or equal to 300 MPa, less than or equal to 250 MPa, less than or equal to 200 MPa, less than or equal to 150 MPa, less than or equal to 100 MPa, less than or equal to 80 MPa, less than or equal to 50 MPa, less than or equal to 40 MPa, less than or equal to 30 MPa, less than or equal to 20 MPa, less than or equal to 10 MPa, less than or equal to 9 MPa, less than or equal to 8 MPa, less than or equal to 7 MPa, less than or equal to 6 MPa, less than or equal to 5 MPa, less than or equal to 4 MPa, less than or equal to 3 MPa, less than or equal to 2 MPa, or less than or equal to 1 MPa. Combinations of the foregoing ranges are possible (e.g., greater than or equal to 1 MPa and less than or equal to 400 MPa, greater than or equal to 20 MPa and less than or equal to 250 MPa, greater than or equal to 6 MPa and less than or equal to 8 MPa). Other ranges are also possible.

In some embodiments, the consolidated biomass has a relatively high density. In certain cases, a relatively high density may advantageously allow the consolidated biomass to be stacked in multiple layers without compromising the structural integrity of consolidated biomass in the bottom layers. In some cases, the consolidated biomass has a density of greater than or equal to 250 kg/m³, greater than or equal to 300 kg/m³, greater than or equal to 400 kg/m³, greater than or equal to 500 kg/m³, greater than or equal to 600 kg/m³, greater than or equal to 700 kg/m³, greater than or equal to 800 kg/m³, greater than or equal to 900 kg/m³, greater than or equal to 1000 kg/m³, greater than or equal to 1100 kg/m³, greater than or equal to 1200 kg/m³, greater than or equal to 1300 kg/m³, greater than or equal to 1400 kg/m³, greater than or equal to 1500 kg/m³, greater than or equal to 1750 kg/m³, greater than or equal to 2000 kg/m³, greater than or equal to 2250 kg/m³, or greater than or equal to 2500 kg/m³. In some embodiments, the consolidated biomass has a density of less than or equal to 2500 kg/m³, less than or equal to 2250 kg/m³, less than or equal to 2000 kg/m³, less than or equal to 1750 kg/m³, less than or equal to 1500 kg/m³, less than or equal to 1400 kg/m³, less than or equal to 1300 kg/m³, less than or equal to 1200 kg/m³, less than or equal to 1100 kg/m³, less than or equal to 1000 kg/m³, less than or equal to 900 kg/m³, less than or equal to 800 kg/m³, less than or equal to 700 kg/m³, less than or equal to 600 kg/m³, less than or equal to 500 kg/m³, less than or equal to 400 kg/m³, less than or equal to 300 kg/m³, or less than or equal to 250 kg/m³. Combinations of the foregoing ranges are possible (e.g., greater than or equal to 700 kg/m³ and less than or equal to 1500 kg/m³, greater than or equal to 500 kg/m³ and less than or equal to 2000 kg/m³, greater than or equal to 250 kg/m³ and less than or equal to 2500 kg/m³). Other ranges are also possible.

In some cases, the consolidated biomass (i.e., densified biomass) has a density that is at least 2 times, at least 5 times, at least 10 times, at least 15 times, at least 20 times, at least 30 times, at least 40 times, at least 50 times, or at least 100 times, greater than a density of the unconsolidated biomass (e.g., the biomass prior to being consolidated). In certain embodiments, a density of the consolidated biomass is 2 to 5 times, 2 to 10 times, 2 to 15 times, 2 to 20 times, 2 to 30 times, 2 to 40 times, 2 to 50 times, 5 to 10 times, 5 to 15 times, 5 to 20 times, 5 to 30 times, 5 to 40 times, 5 to 50 times, 10 to 15 times, 10 to 20 times, 10 to 30 times, 10 to 40 times, 10 to 50 times, 15 to 20 times, 15 to 30 times, 15 to 40 times, 15 to 50 times, 20 to 30 times, 20 to 40 times, 20 to 50 times, 30 to 40 times, 30 to 50 times, or 40 to 50 times greater than a density of the unconsolidated biomass.

In some embodiments, the biomass may be molded into specific shapes during and/or after consolidation. For example, the shape of the consolidated biomass may be substantially cubic, spherical, ellipsoidal, cylindrical, a triangular prism, a rectangular prism, a hexagonal prism, an octagonal prism, a truncated icosahedron, or any other regular three-dimensional shape. In certain embodiments, the consolidated biomass may have an irregular three-dimensional shape. The shape of the consolidated biomass may be designed such that multiple units of consolidated biomass may be stacked and/or stored with minimal space between the units of biomass (e.g., a relatively high packing efficiency, for example, greater than or equal to 80%, greater than or equal to 85%, greater than or equal to 90%, greater than or equal to 95%, or greater than or equal to 99%). In some cases, the biomass may be molded into briquettes and/or blocks during or after consolidation. In some cases, molding the consolidated biomass into briquettes and/or blocks may be useful for efficiently packing the briquettes and/or blocks (e.g., stacking) for transport, handling, storing, and/or monitoring. According to some embodiments, the consolidated biomass (e.g., briquettes and/or blocks) may be engineered to withstand compressive and/or shear loads. In some embodiments, the consolidated biomass (e.g., briquettes and/or blocks) comprise one or more structural features to facilitate stacking and/or withstand compressive and/or shear loads. A non-limiting example of a suitable structural feature is a shear key. In some embodiments, molding the biomass into an object of known dimensions (e.g., a briquette and/or a block) may facilitate encapsulation of the consolidated biomass and allow a seal (e.g., a hermetic seal) to be achieved. According to some embodiments, the shape of the consolidated biomass may be selected to facilitate efficient packing of two or more consolidated biomass units. In some cases, flowability and/or the ability to bag units of consolidated biomass is desired. In certain cases, the ability to stack consolidated biomass units with little-to-no-void space between units, e.g., on a palette as described elsewhere herein is desired. In some embodiments, certain shapes for the consolidated biomass may be chosen due to the ability to form the consolidated biomass into such shapes. For instance, in some embodiments, the consolidated biomass may be extruded using an extrusion line such that the consolidated biomass formed therefrom are cylindrical. Cylindrically shapes consolidated biomass units may be mechanically stable, and thus may facilitate easy storage without mechanical breakdown of the consolidated biomass units.

FIG. 2C shows an exemplary embodiment of consolidated biomass in the shape of a block 254. The block 254 has a first dimension 256, a second dimension 258, and a third dimension 260, which may be any of a variety of sizes. In some cases, the first dimension, the second dimension, and/or the third dimension of a briquette and/or block may each independently be greater than or equal to 1 cm, greater than or equal to 2 cm, greater than or equal to 3 cm, greater than or equal to 4 cm, greater than or equal to 5 cm, better than or equal to 6 cm, greater than or equal to 7 cm, greater than or equal to 8 cm, greater than or equal to 9 cm, greater than or equal to 10 cm, greater than or equal to 20 cm, greater than or equal to 30 cm, greater than or equal to 40 cm, greater than or equal to 50 cm, greater than or equal to 80 cm, greater than or equal to 100 cm, greater than or equal to 120 cm, or greater than or equal to 150 cm. In accordance with some embodiments, the first dimension, the second dimension, and/or the third dimension of a briquette and/or block may each independently be less than or equal to 150 cm, less than or equal to 120 cm, less than or equal to 100 cm, less than or equal to 80 cm, less than or equal to 50 cm, less than or equal to 40 cm, less than or equal to 30 cm, less than or equal to 20 cm, less than or equal to 10 cm, less than or equal to 9 cm, less than or equal to 8 cm, less than or equal to 7 cm, less than or equal to 6 cm, less than or equal to 5 cm, less than or equal to 4 cm, less than or equal to 3 cm, less than or equal to 2 cm, or less than or equal to 1 cm. Independently for each dimension, combinations of the foregoing ranges are possible (e.g., greater than or equal to 1 cm and less than or equal to 150 cm, greater than or equal to 1 cm and less than or equal to 50 cm, greater than or equal to 10 cm and less than or equal to 30 cm, greater than or equal to 3 cm and less than or equal to 6 cm). Other ranges are also possible. Note that, in some cases, each of the dimensions may be equivalent. In other cases, not all or none of the dimensions may be equivalent to each other. In certain instances, two or more briquettes may be combined (e.g., within a sealed container or bag, etc.) to form a larger unit.

According to some embodiments, one or more of the dimensions of the consolidated biomass briquettes may be relatively small (e.g., less than or equal to 10 cm, 5 cm, 2 cm). The relatively small dimensions of the consolidated biomass briquettes may enable flexible handling and/or transport of the consolidated biomass briquettes. The small dimensions, in some such cases, may lead to increased surface-area-to-volume ratios of the consolidated biomass briquettes, relative to the initial size of the biomass, which may improve throughput and/or homogeneity of other processing steps (e.g., adding an additive as described elsewhere herein). In some embodiments, the small dimensions may facilitate subsequent processing steps, such as the individual encapsulation of each briquette and/or sterilization of the briquettes. Moreover, in some such cases, due to the small dimensions of the consolidated biomass briquettes, when the consolidated biomass briquettes are stored (e.g., stacked and/or buried), any failure (e.g., misconfiguration, structural breakdown, and/or rupture of an encapsulating layer) of a single consolidated biomass briquette may have minimal impact. That is, the failure of one consolidated biomass briquette may be relatively inconsequential to the totality of the briquettes, on a wt. % basis. That is, as discussed elsewhere, the relatively small dimensions of the briquettes may enable the storage of a relatively large plurality of briquettes (e.g., greater than or equal to 1000 briquettes, greater than or equal to 1 million briquettes, greater than or equal to 1 billion briquettes, or other amounts as disclosed elsewhere herein), and thus the mechanical failure of one briquette may account for a small wt. % of the total stored biomass (e.g., less than or equal to 0.001 wt. %, less than or equal to 0.0001 wt. %, or less than or equal to 0.00001 wt. %). In some cases, mechanical failure of one consolidated biomass briquette may release CO₂ and/or CH₄ in an amount of less than or equal to 1 ppm, less than or equal to 0.5 ppm, less than or equal to 0.1 ppm, less than or equal to 0.05 ppm, less than or equal to 0.01 ppm, less than or equal to 0.005 ppm, less than or equal to 0.001 ppm, less than or equal to 0.0005 ppm, less than or equal to 0.0001 ppm, less than or equal to 0.00005 ppm, or less than or equal to 0.00001 ppm.

In some embodiments, processing the biomass comprises encapsulating the biomass in one or more layers (e.g., one or more layers comprising a polymeric material). In some embodiments, the one or more layers have a relatively low gas transmission rate of water vapor and/or oxygen. Accordingly, in certain cases, encapsulating the biomass in the one or more layers may advantageously reduce or eliminate formation of carbon-containing gases (e.g., CO₂, CH₄) by reducing or preventing introduction of microbes, water (e.g., liquid water, water vapor), and/or oxygen into the encapsulated biomass, thereby delaying, reducing, or eliminating biomass decomposition. In some embodiments, low water and/or oxygen transmission rates of the one or more layers may inhibit and/or completely prevent microbial growth of biomass encapsulated therein. In certain cases, encapsulating the biomass in the one or more layers may advantageously reduce or eliminate release of any carbon-containing gases (e.g., CO₂, CH₄) that are produced by decomposition of the encapsulated biomass due to the low gas transmission rate of the one or more layers. In addition, in some such cases wherein the biomass may begin to decompose and form CO₂ and/or CH₄, the low gas transmission rate of the one or more layers may lead to increased CO₂ and/or CH₄ levels in the encapsulated biomass, shifting the equilibrium of CO₂ and/or CH₄ production and thereby slowing the rate of decomposition.

Encapsulating, in accordance with some embodiments, may comprise surrounding the biomass with one or more layers (e.g., one or more layers comprising a polymeric material). In certain embodiments, encapsulating the biomass comprises coating, wrapping, shrink fitting, spraying, brushing, dip-coating and/or otherwise forming one or more layers (e.g., one or more layers comprising a polymeric material) around the consolidated biomass. In certain embodiments, encapsulating the biomass comprises wrapping with a membrane. In some embodiments, encapsulating excludes wrapping. In certain embodiments, encapsulating the biomass comprises In some embodiments, the one or more layers comprise a substantially conformal coating. In certain embodiments, the one or more layers comprise one or more layers formed around the consolidated biomass (e.g., via wrapping, shrink fitting, spraying, brushing, and/or dip-coating). In certain embodiments, the one or more layers comprise a pre-engineered envelope (e.g., a bag or container). In some instances, as opposed to a wrapping process that may require complex machinery and/or have more difficulty forming a hermetic seal, the consolidated biomass may be inserted into a pre-formed envelope (e.g., a bag or container). In certain instances, the pre-formed envelope (e.g., bag or container) may comprise a polymeric material. According to some embodiments, encapsulating comprises inserting the biomass into a pre-formed envelope (e.g., a pre-formed bag) and sealing the envelope.

According to some embodiments, individual units of biomass (e.g., briquettes, blocks, cylinders, pellets, etc.) may be separately encapsulated, e.g., as an alternative and/or in addition to wrapping, bagging, or otherwise encapsulating a grouping of multiple units of biomass together. In some embodiments, encapsulating comprises individually encapsulating biomass units of a plurality of biomass units produced during encapsulation of the biomass. In some embodiments, encapsulating comprises encapsulating groups of biomass units, each group of biomass units comprising a portion of all biomass units produced during encapsulation of the biomass. Individually encapsulating single units of biomass can provide certain advantages, such as better isolation from oxygen/water vapor, the ability to make or maintain a desired formed shape of the biomass unit, which may allow the biomass units to be easier to stack, transport, handle, store, and/or monitor. For example, in some embodiments, individually encapsulating individual biomass units may mechanically stabilize the biomass within the individual units, thereby preventing loss of biomass and/or biomass unit form or mechanical integrity during further transport, stacking, or handling, which may also improve accuracy of any carbon measurement and/or tracking of the system. Furthermore, with individual biomass unit encapsulation, any breach or damage of an encapsulating barrier layer can expose less biomass to a loss of aseptic conditions, since a smaller quantity of biomass is contained within each capsule, as opposed to such capsules containing multiple consolidated biomass units.

Additionally, individual encapsulation of biomass units, according to certain embodiments herein, may further advantageously allow for the labelling of individual biomass units. For instance, the one or more layers encapsulating each individual biomass unit may facilitate a corresponding label on each biomass unit, e.g., by adhering a label to the one or more layers, printing a label on the one or more labels, etc., as described in more detail herein, permitting more discrete resolution for tracking, leakage detection, and/or integrity monitoring. Individually encapsulated consolidated biomass units may also, in certain embodiments, be grouped together and further encapsulated in a secondary, tertiary, quaternary, etc. encapsulating step with an encapsulating material that may different or the same as the primary encapsulating material to add further protection against air, water, microbes, etc.

In some embodiments, the one or more encapsulating layers form a hermetic seal around the one or more biomass units (e.g., consolidated biomass units) contained therein. According to certain embodiments, the one or more encapsulating layers hermitically encase the one or more biomass units (e.g., consolidated biomass units). For instance, one or more layers encapsulating the biomass may comprise a material having a relatively low oxygen transmission rate. In some embodiments, an oxygen transmission rate may be measured by an ASTM D3985-17 standard test. In some embodiments, an oxygen transmission rate of one or more layers may be less than or equal to 1 cc/m²/24 hours, less than or equal to 0.9 cc/m²/24 hours, less than or equal to 0.8 cc/m²/24 hours, less than or equal to 0.7 cc/m²/24 hours, less than or equal to 0.6 cc/m²/24 hours, less than or equal to 0.5 cc/m²/24 hours, less than or equal to 0.4 cc/m²/24 hours, less than or equal to 0.3 cc/m²/24 hours, less than or equal to 0.2 cc/m²/24 hours, less than or equal to 0.1 cc/m²/24 hours, less than or equal to 0.09 cc/m²/24 hours, less than or equal to 0.08 cc/m²/24 hours, less than or equal to 0.07 cc/m²/24 hours, less than or equal to 0.06 cc/m²/24 hours, less than or equal to 0.05 cc/m²/24 hours, less than or equal to 0.04 cc/m²/24 hours, less than or equal to 0.03 cc/m²/24 hours, less than or equal to 0.02 cc/m²/24 hours, less than or equal to 0.01 cc/m²/24 hours, less than or equal to 0.005 cc/m²/24 hours, or less than or equal to 0.001 cc/m²/24 hours. In some embodiments, each individual encapsulating layer of the one of more layers may have any of the oxygen transmission rates disclosed herein. In some embodiments, the one or more layers may include two layers, three layers, four layers, etc. as described elsewhere herein, where the oxygen transmission rate of the one or more layers (e.g., the two layers, three layers, four layers, and so forth) in totality may be any of the foregoing ranges or lower.

Encapsulating the biomass may be performed using any suitable device. Non-limiting examples of suitable devices include coating machines (e.g., spray-coating machines, dip-coating machines), wrapping machines, shrink fitting machines, and automated bagging machines. Those of ordinary skill and art will recognize other suitable methods and devices for encapsulating biomass.

A step of encapsulating the biomass may occur before and/or after a step of sterilizing the biomass. Accordingly, encapsulating the biomass may comprise encapsulating sterilized and/or unsterilized biomass. In certain embodiments, sterilizing the biomass may comprise a plurality of sterilizing steps, and a step of encapsulating the biomass may be performed between two or more of the plurality of sterilizing steps. As a non-limiting, illustrative example, a step of encapsulating the biomass may be performed after a first step of sterilizing the biomass and before a second step of sterilizing the biomass. That is, the biomass may be at least partially sterilized prior to encapsulation and then may be further sterilized after encapsulation. In certain cases, the first sterilizing step and the second sterilizing step may utilize different sterilization methods (e.g., the first sterilizing step may use exposure to UV radiation and the second sterilizing step may use heat drying). In certain cases, the first sterilizing step and the second sterilizing step may utilize the same sterilization method. In some instances where the first sterilizing step and the second sterilizing step utilize the same sterilization method, one or more parameters (e.g., sterilization temperature, sterilization time) may be changed.

In certain embodiments, a step of encapsulating the biomass occurs within a relatively short time period after a step of sterilizing the biomass and/or a step of consolidating the biomass. In some cases, for example, an encapsulating step begins less than 60 minutes, 45 minutes, 30 minutes, 20 minutes, 15 minutes, 10 minutes, 5 minutes, 2 minutes, 1 minute, 45 seconds, 30 seconds, 10 seconds, 5 seconds, or 0 seconds after completion of a sterilizing step and/or completion of a consolidating step. In some cases, the time between completion of a sterilizing step and/or a consolidating step and initiation of an encapsulating step is in a range from 0 to 5 seconds, 0 to 10 seconds, 0 to 30 seconds, 0 seconds to 45 seconds, 0 seconds to 1 minute, 0 seconds to 2 minutes, 0 seconds to 5 minutes, 0 seconds to 10 minutes, 0 seconds to 15 minutes, 0 seconds to 20 minutes, 0 seconds to 30 minutes, 0 seconds to 45 minutes, 0 seconds to 60 minutes, 10 to 30 seconds, 10 seconds to 45 seconds, 10 seconds to 1 minute, 10 seconds to 2 minutes, 10 seconds to 5 minutes, 10 seconds to 10 minutes, 10 seconds to 15 minutes, 10 seconds to 20 minutes, 10 seconds to 30 minutes, 10 seconds to 45 minutes, 10 seconds to 60 minutes, 30 seconds to 1 minute, 30 seconds to 2 minutes, 30 seconds to 5 minutes, 30 seconds to 10 minutes, 30 seconds to 15 minutes, 30 seconds to 20 minutes, 30 seconds to 30 minutes, 30 seconds to 45 minutes, 30 seconds to 60 minutes, 1 to 5 minutes, 1 to 10 minutes, 1 to 15 minutes, 1 to 20 minutes, 1 to 30 minutes, 1 to 45 minutes, 1 to 60 minutes, 5 to 10 minutes, 5 to 15 minutes, 5 to 20 minutes, 5 to 30 minutes, 5 to 45 minutes, 5 to 60 minutes, 10 to 20 minutes, 10 to 30 minutes, 10 to 45 minutes, 10 to 60 minutes, 20 to 30 minutes, 20 to 45 minutes, 20 to 60 minutes, 30 to 45 minutes, 30 to 60 minutes, or 45 to 60 minutes.

In certain embodiments, a step of encapsulating the biomass occurs within a relatively short distance from a step of drying and/or sterilizing the biomass and/or a step of consolidating the biomass. For example, in some instances, a sterilization output location (e.g., a location where sterilized biomass is deposited following completion of sterilization) and/or a consolidation output location (e.g., a location where consolidated biomass is deposited following completion of consolidation) is within a relatively short distance from an encapsulation input location (e.g., a location where biomass is deposited for encapsulation). In some cases, a distance between a sterilization output location and/or a consolidation output location and an encapsulation input location is about 60 meters or less, 50 meters or less, 40 meters or less, 30 meters or less, 20 meters or less, 10 meters or less, 8 meters or less, 5 meters or less, 2 meters or less, 1 meter or less, 0.5 meters or less, 0.1 meters or less, or 0 meters. In some cases, the distance between a sterilization output location and/or a consolidation output location and an encapsulation input location is in a range from 0 to 0.1 meters, 0 to 0.5 meters, 0 to 1 meter, 0 to 2 meters, 0 to 5 meters, 0 to 8 meters, 0 to 10 meters, 0.1 to 0.5 meters, 0.1 to 1 meters, 0.1 to 2 meters, 0.1 to 5 meters, 0.1 to 8 meters, 0.1 to 10 meters, 0.5 to 1 meters, 0.5 to 2 meters, 0.5 to 5 meters, 0.5 to 8 meters, 0.5 to 10 meters, 1 to 2 meters, 1 to 5 meters, 1 to 8 meters, 1 to 10 meters, 2 to 5 meters, 2 to 8 meters, 2 to 10 meters, 5 to 10 meters, 8 to 10 meters, 0 to 20 meters, 0 to 30 meters, 0 to meters, 0 to 50 meters, 0 to 60 meters, 10 to 20 meters, 10 to 30 meters, 10 to 40 meters, 10 to 50 meters, 10 to 60 meters, 20 to 30 meters, 20 to 40 meters, 20 to 50 meters, 20 to 60 meters, 30 to 40 meters, 30 to 50 meters, 30 to 60 meters, 40 to 50 meters, 40 to 60 meters, or 50 to 60 meters. In some embodiments, a device for encapsulating the biomass may be connected to a device for sterilizing and/or consolidating the biomass (e.g., such that biomass may be directly deposited from a sterilization and/or consolidation output location to an encapsulation input location). In certain cases, a relatively short distance between a sterilization output location and/or a consolidation output location and an encapsulation input location may advantageously reduce or prevent introduction of microbes between the sterilizing and encapsulating steps.

In some embodiments, encapsulating the biomass may be performed in an at least partially enclosed facility. In certain embodiments, encapsulating the biomass may be performed in a wholly enclosed (e.g., indoor) facility. In some cases, performing the encapsulating step in a wholly enclosed (e.g., indoor) facility may advantageously reduce or eliminate contamination during the encapsulation process. In certain embodiments, a step of encapsulating the biomass occurs in the same facility as a step of consolidating the biomass. In certain embodiments, a step of encapsulating the biomass occurs in the same facility as a step of sterilizing the biomass. In certain embodiments, a step of encapsulating the biomass occurs in the same facility as a step of comminuting the biomass.

FIG. 2D shows schematic illustrations of non-limiting examples of encapsulation. The top portion of FIG. 2D shows a schematic illustration of a non-limiting example of encapsulation, where consolidated biomass 254 is uniformly and conformally coated with layer 262 (e.g., via wrapping, shrink fitting, spraying, brushing, and/or dip-coating). The bottom portion of FIG. 2D shows a schematic illustration of a non-limiting example of encapsulation, where consolidated biomass 254 is inserted into pre-formed bag or envelope 262. FIG. 2E shows a cross-sectional view of consolidated biomass 254 and layer 262.

The one or more layers may comprise any of a variety of materials. In certain embodiments, the one or more layers for encapsulating the biomass may comprise a polymeric material. In some embodiments, the polymeric material has relatively low permeability to water and/or oxygen, relatively high ductility (e.g., to facilitate formation of hermetic seals), and/or a relatively long half-life of decay. In some cases, the polymeric material may comprise a thermoplastic polymer, such as polyethylene terephthalate (PET), polypropylene (PP), high-density polyethylene (HDPE), polyvinyl chloride (PVC), polystyrene (PS), polyethylene (PE), poly(lactic acid) (PLA), polyamide-6 (PA6), polyethylene naphthalate (PEN), poly(m-xylylene adipamide) (MXD6), polyvinyl alcohol (PVOH), ethylene vinyl alcohol (EVOH), polyvinylidene chloride (PVDC), and/or other thermoplastic polymers. In certain cases, it may be particularly advantageous to use a polymeric material comprising PET. In certain embodiments, the PET is biaxially oriented PET (BoPET). In some cases, the polymeric material comprises a recycled polymeric material. In certain cases, using a recycled polymeric material (e.g., recycled PET) may have the benefit of minimizing the amount of waste plastic that enters the environment and/or reducing the need for generating more plastic. Moreover, in cases wherein the one or more layers comprise a polymeric material, the polymeric material may comprise carbon and thus may further contribute to carbon sequestration. In certain embodiments, at least one (and, in some cases, each) layer of the one or more layers may comprise two or more polymeric materials.

In some embodiments, at least one of the one or more encapsulating layers comprises a non-thermoplastic and/or curable polymer, and/or bio-based, non-synthetic polymer or resin, and/or highly viscous adhesive materials (e.g. bitumen, pitch, asphalt, etc.), and methods for encapsulating biomass with such encapsulating materials may include, without limitation, any suitable methods described above for thermoplastic encapsulating materials. Suitable materials may include, without limitation, thermoset polymers and natural polymers and resins such as amber. Such materials, additionally can be cured or otherwise formed into a solid encapsulating layer through, for example, non-thermally-driven phase change processes, such as crosslinking or chain extension mechanisms, solvent evaporation, etc. Such materials are or contain polymers and/or macromolecules that form films, layers, networks, etc. as they cure, for example through the creation of covalent chemical bonds between different polymer chains in a cross linking or covalent monomer/macromer addition via chain extension mechanisms. Such covalent chemical bonds can be induced through a number of different pathways including free radical polymerization, vulcanization of rubbers or other elastomers or direct cross-linking between individual reactive chemical moieties on the polymer chains. An insoluble network of these types of materials may form through means of the curing process that may be functionally irreversible and thus able to create very long-lasting and durable materials for stable encapsulation. In certain cases, the formed polymeric network is extremely resistant to heat degradation or chemical attack, rendering the biomass units encapsulated with such materials stably encased for extended sequestration periods. For example, perfectly preserved insects have been found inside amber that is 45 million years old demonstrating the ability of these types of cross-linked materials to prevent decomposition of organic material encases therein over very long periods of time.

According to some embodiments, thermoset materials suitable for use in at least one of the one or more encapsulation layers can be tailored to be mechanically robust and/or to provide a barrier for water and gas transport, desirable properties for encapsulating biomass to prevent decomposition. In many commercial applications, packaging materials need to be removable to access the item being packaged (e.g., food, pharmaceutical product), and thus thermoset materials are not typically suitable for use. However, in certain embodiments of encapsulating consolidated biomass for carbon removal purposes as described herein, since one goal can be to have the biomass material stay stably encased for prolonged periods, e.g., thousands of years in some cases, this may be achieved by, for example, using thermosets for forming at least one encapsulating layer encasing biomass that are irreversibly chemically crosslinked upon curing. In some such embodiments, once the thermoset polymer is applied to the biomass and cured, it can provide long-term or essentially indefinite protection from ingression of water and/or oxygen. Coatings of thermoset polymers can also be highly conformal due to the nature of resins often being liquid or formable prior to curing.

Relevant thermoset chemistries that can be utilized for the purpose of encasing biomass (e.g., consolidated biomass, densified biomass) to prevent decomposition include polyurethanes, formaldehyde-based polymers, cyanate esters, polyimides, and epoxies. Such polymers can be applied through any appropriate technique, such as via dipping, painting, or spraying onto biomass, for example, consolidated (e.g., densified) biomass, as described elsewhere herein. The thermoset material may also in some cases be applied as a two-part formulation such as in the case of an epoxy. In certain such cases, an epoxide resin can be mixed with a hardener (e.g., an amine) that cures to form an epoxy encapsulating barrier layer. This may be done, for example, by spraying, dipping or painting the biomass with the first component followed by spraying, dipping or painting the biomass with the second component. The curing or cross-linking of polymers or pre-polymers for the purpose of encapsulating biomass may take place through, for example, heating, radiation, application of a catalyst material (e.g., a hardener), and/or application of pressure.

By way of example, polyurethanes are a set of polymer chemistries that can be either thermosets or thermoplastics. In some cases, this disclosure describes using thermoset polyurethanes that are crosslinked as the material for at least one or more encapsulating layers, providing durability and strong mechanical properties including resistance to abrasion. Polyurethanes are known to have a urethane linkage and are usually synthesized by reaction of an alcohol with an isocyanate. The alcohol may be a polyol and the structure of the polyol can contribute to a branched and cross-linked structure that is characteristic of a thermoset material. For example, short chain, low molecular weight polyols can react with aromatic isocyanates to provide highly structured and rigid polyurethanes. To apply these types of materials as an encapsulating material, the reaction may be performed while the monomers or pre-polymers are coated on or surrounding the material to be encapsulated. One way this may be done is by mixing the materials and then immediately coating them onto the biomass and allowing the solvent to evaporate while the curing reaction takes place. In such an embodiment, what would be left is a rigid thermoset polyurethane completely or substantially encasing the consolidated biomass unit(s).

As another example, phenol formaldehyde polymers can be formed through the reaction of phenol with formaldehyde to form a polymer network. The reaction can in certain cases be a two-step process, with an initial step involving the phenol and formaldehyde being brought into in contact with each other, e.g., where the molar ratio of formaldehyde to phenol is less than 1, to produce a fluent or conformable prepolymer which can then then be further cured by heating the prepolymer while adding more formaldehyde. Fully cured phenol formaldehyde resins can provide thermosetting polymer networks that are mechanically robust and resistant to degradation-well-suited for stably encapsulating biomass within at least one of the one or more layers, according to some embodiments. The polymerization/curing reaction may proceed as a step-growth polymerization that produces methylene bridge linkages between the phenol groups. In some cases, when the molar ratio of formaldehyde to phenol reaches one, the system is fully crosslinked as each phenol group is theoretically linked at that point. These types of resins are very rigid and mechanically strong and are used in everything from billiard balls to countertops.

Yet another example of a suitable or potentially suitable encapsulating material (e.g., for encapsulating consolidated biomass, densified biomass) are melamine/melamine-formaldehyde polymers. Such polymers are another class of thermosetting polymers, in this case using formaldehyde as one of the components, that are suitable for use as the material in at least one of the one or more encapsulating layers, in some embodiments. To produce such polymers, the formaldehyde may be condensed with melamine to produce a hydroxymethyl compound. That hydroxymethyl species can then be heated in the presence of an acid to form linkages through further condensation and cross linking, thereby resulting in a thermoset polymer. Such materials have been used to make dishes, countertops, flooring and in other applications where their water resistance and strong mechanical properties make them advantageous. These properties can similarly be appropriate and advantageous in serving as a barrier to water or gases getting into encapsulated, stored biomass.

Another suitable or potentially suitable class of thermosetting polymers for use in the disclosed methods and for biomass encapsulation are cyanate esters. These materials have the formula R—O—C≡N where R is an organyl group. The cyanate esters can be cured through heating or with a catalyst to produce thermoset materials that, for certain species, can have very high toughness and high glass transition temperatures. The flexibility and relative ease of curing can be advantageous for the application of such materials as a protective coating or layer for stored biomass (e.g., consolidated biomass, densified biomass, etc.). At least certain such materials may be applied as a fluent, uncured or only partially cured polymer network and then heated to instigate or complete the curing process without, in certain cases, needing to add additional components that might require mixing or management of inhomogeneity.

Epoxies are another suitable or potentially suitable class of thermosetting materials for use in at least one of the one or more layers, in some embodiments. Epoxies are formulated and supplied typically as prepolymers, macromers, or polymers that contain epoxide groups that can undergo catalytic homopolymerization and/or react with small molecule or monomer additives, and/or other macromers, pre-polymers, polymers with chemical groups reactive with epoxide groups, such as amines, acids, phenols, alcohols or thiols. Such additives serve as hardeners by introducing cross-linking moieties throughout the polymer. Epoxy chemistry is quite diverse and can result in a wide range of properties, but many and typical epoxies have very good mechanical strength properties and high chemical and thermal resistance. The curing process can be slow in some instances, sometimes taking weeks to reach the full mechanical properties, but this is dependent on the specific reaction that is utilized as well as the curing conditions.

Additionally, according to some embodiments, another exemplary non-thermoplastic polymer class that may be suitable as the encapsulating material for at least one of the one or more encapsulating layers in the context of the present disclosure are polyimides, which can be formed, for example, through reaction of a dianhydride and a diamine or between a dianhydride and a diisocyanate. In either of these synthetic pathways, the resulting material is, in typical embodiments, a polymer network that is relatively lightweight while having suitable mechanical and thermal properties. Polyimides are also typically resistant to flame combustion which could be advantageous in protecting encapsulated biomass from the risk of fire at any point in storage or transportation.

Another alternative group of materials suitable or potentially suitable for at least certain embodiments of the disclosed encapsulating materials are natural (i.e., found in nature and/or non-synthetically produced) resins, e.g., cross-linking resins, such as plant-based resins or sap (e.g., amber). Certain such materials, e.g., amber, are able to resist degradation for millennia, making them attractive for applications where a one goal can be to prevent intrusion of water or oxygen into the stored biomass over very long periods of time. This class also encompasses materials such as natural rubbers (e.g., latex/polyisoprene) (when vulcanized); balsams; copal; kauri gum; rosin; shellac; others; and resin varnishes made from these through addition of drying oils (such as linseed oil, tung oil, and walnut oil that contain high levels of polyunsaturated fatty acids), and solvents, which cure or harden upon drying. In certain embodiments, two or more of these types of materials could be combined to improve the overall encapsulating layer(s) properties and/or could be combined with one or more of the previously-described synthetic thermosetting and/or thermoplastic encapsulating materials for tuning properties of a composite or polymer mixture-based encapsulating material.

In one set of embodiments, biomass encapsulation may occur initially with a material that is particularly impermeable to moisture (e.g., amber) and, sequentially or in combination, another material that is more mechanically robust, resilient, shock-absorbing, etc. (e.g., a vulcanized natural rubber, a thermoplastic polymer, etc.) to protect the encapsulated biomass from mechanical abrasion or damage. In certain embodiments, it may be advantageous to encapsulate the biomass with a mixture of thermoset polymers as well as with other materials, such as thermoplastic polymers, to access advantageous properties of multiple classes of materials. Certain thermoset materials can become brittle, putting them at risk for fracturing during transport and handling of an encased biomass block. However, by mixing other materials such as thermoplastic polymers, oligomers, or other small (e.g. plasticizer) molecules, the encapsulating material may be rendered less brittle while still maintaining the longevity of a cross-linked thermoset polymer. Suitable properties can be measured, for example, by using a Charpy Impact test that provides a stress/strain curve demonstrating the energy that is required to fracture the candidate material(s).

In some embodiments, at least one layer of the one or more encapsulating layers is directly adjacent to the consolidated biomass. That is, in certain embodiments, at least one layer of the one or more layers is in direct physical contact with at least a portion of the consolidated biomass. In some such embodiments, no intervening layers or components may be present between the one or more layers and the consolidated biomass. In other embodiments, one or more intervening layers or components may be present between the one or more layers and the consolidated biomass. In certain cases, the presence of at least one layer of the one or more layers directly adjacent to the consolidated biomass may advantageously maximize protection of the consolidated biomass against exposure to water, oxygen, and/or microbes.

In some cases, at least one (and, in some cases, each) layer of the one or more encapsulating layers has a thickness of greater than or equal to 100 nm, greater than or equal to 500 nm, greater than or equal to 1 microns, greater than or equal to 10 microns, greater than or equal to 50 microns, greater than or equal to 100 microns, greater than or equal to 250 microns, greater than or equal to 500 microns, greater than or equal to 1 mm, greater than or equal to 5 mm, greater than or equal to 8 mm, or greater than or equal to 10 mm. In some cases, the thickness of at least one (and, in some cases, each) layer of the one or more layers is less than or equal to 10 mm, less than or equal to 8 mm, less than or equal to 5 mm, less than or equal to 3 mm, less than or equal to 2 mm, less than or equal to 1 mm, less than or equal to 500 microns, less than or equal to 250 microns, less than or equal to 100 microns, less than or equal to 50 microns, less than or equal to 10 microns, less than or equal to 500 nm, or less than or equal to 100 nm. Combinations of the foregoing ranges are possible (e.g., greater than or equal to 100 nm and less than or equal to 10 mm). Other ranges are also possible. In some instances, using one or more layers with relatively high thicknesses may maintain the integrity of the one or more layers even if a portion of the one or more layers degrades (e.g., by exposure to UV radiation and/or abrasion during transport).

In some cases, a total thickness of the one or more encapsulating layers is greater than or equal to 100 nm, greater than or equal to 500 nm, greater than or equal to 1 microns, greater than or equal to 10 microns, greater than or equal to 50 microns, greater than or equal to 100 microns, greater than or equal to 250 microns, greater than or equal to 500 microns, greater than or equal to 1 mm, greater than or equal to 5 mm, greater than or equal to 8 mm, greater than or equal to 10 mm, greater than or equal to 15 mm, greater than or equal to 20 mm, or greater than or equal to 25 mm. In some cases, a total thickness of the one or more layers is less than or equal to 25 mm, less than or equal to 20 mm, less than or equal to 15 mm, less than or equal to 10 mm, less than or equal to 8 mm, less than or equal to 5 mm, less than or equal to 3 mm, less than or equal to 2 mm, less than or equal to 1 mm, less than or equal to 500 microns, less than or equal to 250 microns, less than or equal to 100 microns, less than or equal to 50 microns, less than or equal to 10 microns, less than or equal to 500 nm, or less than or equal to 100 nm. Combinations of the foregoing ranges are possible (e.g., greater than or equal to 100 nm and less than or equal to 25 mm, greater than or equal to 100 nm and less than or equal to 10 mm). Other ranges are also possible.

The one or more encapsulating layers (e.g., one or more layers comprising a polymeric material) may have a relatively high impact resistance to avoid damage during transport and/or handling, for example by abrasion, as well as to maintain structural integrity when stored, in some embodiments. In some cases, the impact resistance of the one or more layers may be measured by an ASTM D256-23el standard Izod impact strength test. In some embodiments, the impact resistance of the one or more layers may be greater than or equal to 20 J/m², greater than or equal to 50 J/m², greater than or equal to 100 J/m², greater than or equal to 150 J/m², greater than or equal to 200 J/m², greater than or equal to 250 J/m², greater than or equal to 300 J/m², greater than or equal to 350 J/m², greater than or equal to 400 J/m², greater than or equal to 450 J/m², greater than or equal to 500 J/m², greater than or equal to 1000 J/m², greater than or equal to 1500 J/m², or greater than or equal to 2000 J/m². In some cases, the impact resistance of the one or more layers may be less than or equal to 2000 J/m², less than or equal to 1500 J/m², less than or equal to 1000 J/m², less than or equal to 500 J/m², less than or equal to 450 J/m², less than or equal to 400 J/m², less than or equal to 350 J/m², less than or equal to 300 J/m², less than or equal to 250 J/m², less than or equal to 200 J/m², less than or equal to 150 J/m², less than or equal to 100 J/m², less than or equal to 50 J/m², or less than or equal to 20 J/m². Combinations of the foregoing ranges are possible (e.g., greater than or equal to 20 J/m² and less than or equal to 2000 J/m²). Other ranges are also possible.

In some cases, the one or more encapsulating layers (e.g., one or more layers comprising a polymeric material) may be substantially impervious to water (e.g., water vapor), oxygen, and/or microbes associated with biomass decomposition, including but not limited to gram-positive bacteria, fungi, and actinomycetes. In accordance with some embodiments, the one or more layers may have a relatively low gas transmission rate of water vapor and/or oxygen, which may reduce or prevent the growth of microbes and the subsequent decomposition of the encapsulated biomass. In some embodiments, an ASTM D3985-17 standard test may be used to measure the gas transmission rate of oxygen of the one or more layers. According to some embodiments, the one or more layers may have a gas transmission rate of oxygen of less than or equal to 10 mol s⁻¹ m⁻², less than or equal to 5 mol s⁻¹ m⁻², less than or equal to 3 mol s⁻¹ m⁻², less than or equal to 1 mol s⁻¹ m⁻², less than or equal to 0.9 mol s⁻¹ m⁻², less than or equal to 0.8 mol s⁻¹ m⁻², less than or equal to 0.7 mol ⁻¹ m⁻², less than or equal to 0.6 mol s⁻¹ m⁻², less than or equal to 0.5 mol s⁻¹ m⁻², less than or equal to 0.4 mol s⁻¹ m⁻², less than or equal to 0.3 mol s⁻¹ m⁻², less than or equal to 0.2 mol s⁻¹ m⁻², less than or equal to 0.1 mol s⁻¹ m⁻², less than or equal to 0.05 mol ⁻¹ m⁻², or less than or equal to 0.01 mol ⁻¹ m⁻². In some embodiments, an ASTM E 96M-22ael standard test may be used to measure the gas transmission rate of water vapor of the one or more layers. According to some embodiments, the one or more layers may have a gas transmission rate of water vapor of less than or equal to 10 mol s⁻¹ m⁻², less than or equal to 5 mol s⁻¹ m⁻², less than or equal to 3 mol s⁻¹ m⁻², less than or equal to 1 mol s⁻¹ m⁻², less than or equal to 0.9 mol s⁻¹ m⁻², less than or equal to 0.8 mol s⁻¹ m⁻², less than or equal to 0.7 mol s⁻¹ m⁻², less than or equal to 0.6 mol s⁻¹ m⁻², less than or equal to 0.5 mol s⁻¹ m⁻², less than or equal to 0.4 mol s⁻¹ m⁻², less than or equal to 0.3 mol s⁻¹ m⁻², less than or equal to 0.2 mol s⁻¹ m⁻², less than or equal to 0.1 mol s⁻¹ m⁻², or less than or equal to 0.05 mol ⁻¹ m⁻², or less than or equal to 0.01 mol ⁻¹ m⁻².

In some embodiments, the one or more encapsulating layers (e.g., one or more layers comprising a polymeric material) have a relatively high coefficient of friction. In certain cases, a relatively high coefficient of friction may advantageously reduce movement of encapsulated biomass during transportation and/or storage in a sequestration site.

In some embodiments, the one or more encapsulating layers (e.g., one or more layers comprising a polymeric material) may further comprise a radiation-absorbing (e.g., UV radiation absorbing) motif and/or molecule that is different from the polymeric material. In some cases, the radiation-absorbing motif and/or molecule may absorb any incoming radiation without producing photoinitiators (e.g., converting the radiation into heat). In some such cases, the radiation-absorbing motif and/or molecule may prevent the one or more layers from degrading, for example, by photooxidation, and thus may extend the lifetime of the one or more layers in the presence of radiation.

In some cases, the one or more encapsulating layers (e.g., one or more layers comprising a polymeric material) may further comprise a radiation-reflecting component. For example, in some cases, the one or more layers may be metallized, wherein a thin film of metal may coat the outer surface of the one or more layers. In some such cases, the thin film of metal may comprise aluminum, gold, nickel, and chromium. In some cases, the thin film of metal may have a thickness less than or equal to 500 microns, less than or equal to 400 microns, less than or equal to 300 microns, less than or equal to 250 microns, less than or equal to 200 microns, less than or equal to 150 microns, or less than or equal to 100microns, less than or equal to 50 microns, or less than or equal to 20 microns. In some such embodiments, the metal film may be present in a thickness sufficient to reflect incoming radiation (e.g., UV radiation) to minimize and/or prevent incident radiation from interacting with the other portions of the one or more layers (e.g., a polymeric material of the one or more layers).

It may be particularly advantageous for the one or more encapsulating layers (e.g., one or more layers comprising a polymeric material) to comprise a material having a relatively long half-life of decay. According to some embodiments, the degradation rate of the material (e.g., polymeric material) of the one or more layers may be measured used an ASTM F1980-21 standard test. In some cases, the material of the one or more layers may structurally degrade after a time of greater than or equal to 100 years, greater than or equal to 250 years, greater than or equal to 500 years, greater than or equal to 750 years, greater than or equal to 1000 years, greater than or equal to 1500 years, greater than or equal to 2000 years, greater than or equal to 2500 years, greater than or equal to 5000 years, or greater than or equal to 10,000 years. In some cases, the material (e.g., polymeric material) of the one or more layers may not measurably degrade during the testing. In some embodiments, the material (e.g., polymeric material) of the one or more layers having a relatively long half-life of decay may result in the one or more layers maintaining their structural integrity for relatively long times and thus encapsulating the biomass for relatively long times. In some such cases, as described elsewhere herein, the encapsulated biomass may be stored to sequester the carbon content of the encapsulated biomass, thereby removing the carbon content, for example, from the atmosphere.

In some cases, more than one encapsulating layer (e.g., more than one layer comprising a polymeric material) may be used. According to some embodiments, the one or more layers may comprise one, two, three, four, five, six, seven, eight, nine, ten, or more layers. FIG. 2F shows a schematic illustration of a non-limiting example of encapsulation, where consolidated biomass 254 is uniformly coated with first layer 262 and second layer 264. FIG. 2G shows a cross-sectional view of consolidated biomass 254, first layer 262, and second layer 264. It will be understood that additional layers are also possible.

In certain cases, at least one (and, in some cases, each) encapsulating layer of the one or more encapsulating layers comprises a polymeric material. In some embodiments, different layers may comprise different polymeric materials. In some such cases, a first layer may comprise a polymeric material that has a relatively low gas transmission rate to oxygen and/or water vapor as described elsewhere herein, whereas the second layer may have a relatively low rate of degradation when exposed to UV radiation and/or a relatively high impact resistance, wherein the second layer may be the outermost layer of the encapsulated biomass. In some such configurations, the first layer may delay and/or prevent decomposition of the biomass and the second layer may delay and/or prevent degradation of the first layer, for example, when the encapsulated biomass is exposed to sunlight (and/or other sources of UV radiation) and/or physical abrasion. In some cases, multiple layers may comprise the same polymeric materials, which may decrease the total gas transmission rate of the one or more layers (e.g., when accounting for all the layers together) to water vapor and/or oxygen. In some cases, multiple layers may enable one or some of the layers to partially degrade while maintaining complete encapsulation of the biomass. In some instances, one or more non-polymeric materials (e.g., oxygen-scavenging compounds) may be present between different layers of the one or more layers. According to some embodiments, each individual encapsulating layer of the one or more encapsulating layers may be included to provide a desired property to the consolidated biomass unit. For example, as described above, in some embodiments, at least one of the one or more layers may have a low water transmission rate. According to some embodiments, at least one of the one or more layers may have a low oxygen transmission rate. In some embodiments, at least one of the one or more layers may have a low CO₂ transmission rate. In some embodiments, at least one of the one or more layers may be at least partially reflective of UV radiation. In some embodiments, at least one of the one or more layers may be relatively mechanically robust, i.e., impact resistant and/or resistant to abrasive degradation. According to some embodiments, one or more of the layers may be reactive with an entity present in the atmosphere, e.g., with water or oxygen. In some such embodiments, it may be desirable to have multiple layers such that any reactive layers may not be exposed to an atmosphere containing a gas with which an interior layer of the multiple layers is reactive. In some embodiments, it may be desirable to have a multilayered structure comprising at least one robust exterior layer resistant to mechanical failure and at least one layer having a low oxygen transmission rate. In some such embodiments, it may further be advantageous to include at least one layer in the multilayered structure that has a low water transmission rate and/or a low CO₂ transmission rate.

In some embodiments, a single encapsulating layer of the one or more encapsulating layers may comprise a plurality of sub-layers laminated (or otherwise combined) into a single layer. In certain embodiments, different sub-layers of the plurality of sub-layers may comprise different polymeric materials (e.g., having different gas transmission rates of oxygen and/or water vapor). In certain embodiments, two or more sub-layers of the plurality of sub-layers may comprise the same polymeric material. In some instances, one or more non-polymeric materials (e.g., oxygen-scavenging compounds) may be present between two or more sub-layers of the plurality of sub-layers.

In some embodiments, encapsulating the consolidated biomass may sufficiently delay and/or prevent decomposition of encapsulated biomass for at least 100 years, at least 500 years, at least 1000 years, at least 1500 years, at least 2000 years, at least 2500 years, at least 5000 years, or at least 10,000 years when the biomass is stored in darkness under standard atmospheric conditions. In some cases, encapsulating the consolidated biomass may sufficiently prevent oxygen and/or water vapor from transporting from a surrounding atmosphere into the encapsulated biomass at rate of greater than or equal to 10 mol s⁻¹ m² for oxygen and greater than or equal to 10 mol s⁻¹ m² for water vapor under standard atmospheric conditions. In certain cases, encapsulating the consolidated biomass units is sufficient to prevent microbial activity and decay of encapsulated biomass for at least 100 years when the biomass is stored in conditions where exposure to light is possible, such as in an above-ground warehouse or similar.

According to some embodiments, processing the biomass may produce an article comprising biomass. In some embodiments, the article further comprises one or more layers (e.g., one or more layers comprising a polymeric material) encapsulating the biomass. In some cases, the one or more layers may be substantially impervious to oxygen, water vapor, and/or carbon dioxide. According to some embodiments, the biomass of the article (e.g. an article that is an encapsulated biomass unit(s)) is substantially free of non-biomass material.

The article may comprise biomass that has been processed by any of the foregoing processing steps, in any order and/or combination. In some embodiments, the biomass is substantially resistant to microbial growth. In some embodiments, the biomass has a sterility assurance level (SAL) of 10⁻¹ or less, 10⁻² or less, 10⁻³ or less, 10⁻⁴ or less, 10⁻⁵ or less, or 10⁻⁶ or less. In some embodiments, the biomass has at least a 1 log reduction, at least a 2 log reduction, at least a 3 log reduction, at least a 4 log reduction, at least a 5 log reduction, or at least a 6 log reduction in the population of a challenge microorganism (e.g., a gram-positive bacterium, a methanogen, and/or a CO₂-producing microbe).

In some embodiments, the consolidated biomass has a relatively high density. In certain embodiments, the consolidated biomass has a density of greater than or equal to 250 kg/m³, greater than or equal to 300 kg/m³, greater than or equal to 400 kg/m³, greater than or equal to 500 kg/m³, greater than or equal to 600 kg/m³, greater than or equal to 700 kg/m³, greater than or equal to 800 kg/m³, greater than or equal to 900 kg/m³, greater than or equal to 1000 kg/m³, greater than or equal to 1100 kg/m³, greater than or equal to 1200 kg/m³, greater than or equal to 1300 kg/m³, greater than or equal to 1400 kg/m³, greater than or equal to 1500 kg/m³, greater than or equal to 1750 kg/m³, greater than or equal to 2000 kg/m³, greater than or equal to 2250 kg/m³, or greater than or equal to 2500 kg/m³. In some embodiments, the biomass has a density of less than or equal to 2500 kg/m³, less than or equal to 2250 kg/m³, less than or equal to 2000 kg/m³, less than or equal to 1750 kg/m³, less than or equal to 1500 kg/m³, less than or equal to 1400 kg/m³, less than or equal to 1300 kg/m³, less than or equal to 1200 kg/m³, less than or equal to 1100 kg/m³, less than or equal to 1000 kg/m³, less than or equal to 900 kg/m³, less than or equal to 800 kg/m³, less than or equal to 700 kg/m³, less than or equal to 600 kg/m³, less than or equal to 500 kg/m³, less than or equal to 400 kg/m³, less than or equal to 300 kg/m³, or less than or equal to 250 kg/m³. Combinations of the foregoing ranges are possible (e.g., greater than or equal to 700 kg/m³ and less than or equal to 1500 kg/m³, greater than or equal to 500 kg/m³ and less than or equal to 2000 kg/m³, greater than or equal to 250 kg/m³ and less than or equal to 2500 kg/m³). Other ranges are also possible.

The consolidated biomass may have any suitable shape. In certain embodiments, the biomass has a substantially cubic, spherical, ellipsoidal, cylindrical, triangular prism, rectangular prism, hexagonal prism, octagonal prism, and/or truncated icosahedron shape. Other shapes are also possible.

The one or more layers may comprise any suitable materials as described in the context of encapsulating biomass above. In some such embodiments, the one or more layers comprise a polymeric material. Non-limiting examples of suitable polymeric materials include PET, BoPET, PP, HDPE, PVC, PS, PE, PLA, PA6, PEN, MXD6, PVOH, EVOH, and PVDC and/or any one or more thermoset, curable, and/or natural resins or coating materials noted previously. In some embodiments, the one or more layers comprise one, two, three, four, five, or more layers. In certain embodiments, a single layer of the one or more layers may comprise a plurality of sub-layers laminated or otherwise combined into a single layer. Different layers of the one or more layers or different sub-layers of the plurality of sub-layers in a single layer may comprise different polymeric materials (e.g., having different gas transmission rates of oxygen and/or water vapor) or the same polymeric material. In some instances, one or more non-polymeric materials (e.g., oxygen-scavenging compounds) may be present between two or more layers of the one or more layers or two or more sub-layers of the plurality of sub-layers in a single layer.

In some cases, the one or more encapsulating layers (e.g., one or more layers comprising a polymeric material) may be substantially impervious to water (e.g., water vapor), oxygen and/or microbes associated with biomass decomposition, including but not limited to gram-positive bacteria, fungi, and actinomycetes. In accordance with some embodiments, the one or more layers may have a relatively low gas transmission rate of water vapor and/or oxygen, which may reduce or prevent the growth of microbes and the subsequent decomposition of the encapsulated biomass. According to some embodiments, the one or more layers may have a gas transmission rate of oxygen of less than or equal to 10 mol s⁻¹ m⁻², less than or equal to 5 mol s⁻¹ m⁻², less than or equal to 3 mol s⁻¹ m⁻², less than or equal to 1 mol s⁻¹ m⁻², less than or equal to 0.9 mol s⁻¹ m⁻², less than or equal to 0.8 mol s⁻¹ m⁻², less than or equal to 0.7 mol s⁻¹ m⁻², less than or equal to 0.6 mol s⁻¹ m⁻², less than or equal to 0.5 mol s⁻¹ m⁻², less than or equal to 0.4 mol s⁻¹ m⁻², less than or equal to 0.3 mol s⁻¹ m⁻², less than or equal to 0.2 mol s⁻¹ m⁻², less than or equal to 0.1 mol s⁻¹ m⁻², less than or equal to 0.05 mol s⁻¹ m⁻², or less than or equal to 0.01 mol s⁻¹ m⁻². In some embodiments, an ASTM E96M-22ael standard test may be used to measure the gas transmission rate of water vapor of the one or more layers. According to some embodiments, the one or more layers may have a gas transmission rate of water vapor of less than or equal to 10 mol s⁻¹ m⁻², less than or equal to 5 mol s⁻¹ m⁻², less than or equal to 3 mol s⁻¹ m⁻², less than or equal to 1 mol s⁻¹ m⁻², less than or equal to 0.9 mol s⁻¹ m⁻², less than or equal to 0.8 mol s⁻¹ m⁻², less than or equal to 0.7 mol s⁻¹ m⁻², less than or equal to 0.6 mol s⁻¹ m⁻², less than or equal to 0.5 mol s⁻¹ m⁻², less than or equal to 0.4 mol s⁻¹ m⁻², less than or equal to 0.3 mol s⁻¹ m⁻², less than or equal to 0.2 mol s⁻¹ m⁻², less than or equal to 0.1 mol s⁻¹ m⁻², or less than or equal to 0.05 mol s⁻¹ m⁻², or less than or equal to 0.01 mol s⁻¹ m⁻².

In certain embodiments, the carbon content of the biomass is quantified prior to storage. In some cases, carbon content of the biomass may be quantified and recorded to comply with a regulatory agency's regulation or policy.

In accordance with some embodiments, a carbon content of encapsulated biomass may be quantified. In certain embodiments, the carbon content of each unit of processed biomass may be quantified to track and/or report the amount of carbon removed from the atmosphere.

According to some embodiments, the carbon content may be quantified with a relatively high accuracy, which may be advantageous when tracking the amount of carbon and/or verifying the amount of carbon removed from the atmosphere. In some cases, the carbon content of the biomass may be determined by mass spectrometry, gravimetric analysis, elemental analysis, and/or dual-energy x-ray imaging. In some embodiments, the carbon content of the biomass may be greater than or equal to 10 wt. %, greater than or equal to 20 wt. %, greater than or equal to 30 wt. %, greater than or equal to 40 wt. %, greater than or equal to 50 wt. %, greater than or equal to 60 wt. %, greater than or equal to 70 wt. %, greater than or equal to 80 wt. %, greater than or equal to 90 wt. %, or greater than or equal to 95 wt. % of the biomass. In some embodiments, taking the wt. % of carbon content in the biomass and multiplying it by the total mass of the biomass may provide the mass of carbon content in the biomass. The total mass of the biomass may be measured according to any suitable method known in the art. In some embodiments, the total mass of the biomass may be measured using a scale (e.g., a standalone scale, a conveyor belt scale, a check weigher scale) or other weighing device. In certain embodiments, biomass (e.g., a volume of biomass to be consolidated into a unit of consolidated biomass) may be weighed prior to consolidation. In certain embodiments, one or more units of consolidated biomass (e.g., briquettes and/or blocks) may be weighed after consolidation. As a non-limiting example, as described elsewhere herein, in some embodiments the biomass may be conveyed along a conveyor belt. In some such embodiments, a check weigher scale may be present at a position along and/or at the end of the conveyor belt such that biomass transported along the conveyor belt may pass over the check weigher scale and have its mass measured.

In some cases, after quantifying the amount of carbon content in a unit of processed (e.g., consolidated and/or encapsulated) biomass, a label (e.g., an RFID label, a barcode, a serial number, etc.) may be applied and/or placed on to the processed biomass, wherein the label may contain information (e.g., weight, type of biomass) about the amount of carbon contained in the unit of biomass. Applying or placing the label may be performed in any of a variety of suitable methods, including printing a label (e.g., using ink or other materials onto an outer layer of a briquette or encapsulating layer), adhering a label, modifying an exterior layer of one or more layers encapsulating the processed biomass (e.g., impressing the label into a compliant outer layer, applying energy such as heat and/or light to induce an optical change in an outer layer, mechanically or chemically etching), other appropriate methods for applying or placing a label, and combinations thereof. As a non-limiting example, a label may be printed directly onto an outer layer of an encapsulated biomass unit. In some embodiments, a label may be printed onto a first side of an article and the second side of an article may include an adhesive for affixing the label to the encapsulated biomass unit.

Applying or placing a label may be advantageous for any of a variety of reasons.

For example, in some cases, the labels may identify and provide information about different units of biomass, for example, if there are variations in the amount of carbon sequestered between each unit of biomass. Moreover, labelling at the outset of the carbon sequestration may allow for more accurate monitoring of the carbon sequestration process over time, in some cases. For instance, a second carbon content of the biomass units may be measured at a later time and then compared to the initial carbon content, and any changes in the carbon content may be tracked by the individual biomass units by using the labels. Identification and monitoring of carbon content may desirably provide the ability to track the amount of carbon sequestered using the processes described herein. According to some embodiments, the labels may be used to track a location of the corresponding biomass unit, e.g., during transport and/or storage. In some embodiments, while it is undesirable for any biomass units to degrade, be damaged, or otherwise leak, labels can facilitate monitoring a carbon content and tracking a location of particular biomass units to desirably aid in determining a location within a storage system where units are predisposed to degrade (e.g., by a weight load distribution, unintended exposure to heat or light, etc.) over a storage period, if any locations exist. Accordingly, such tracking may provide the ability to improve storage facilities over time to avoid or correct locations within the storage system where biomass units are susceptible to degrading.

In some embodiments, the biomass may be palletized for handling, transport, and/or storage. In some cases, the biomass may be comminuted, sterilized, consolidated (e.g., into briquettes and/or blocks), and/or encapsulated before being palletized.

Palletizing the consolidated biomass may be achieved by methods known to those of ordinary skill in the art, for example, by stacking two or more units of consolidated biomass in an orderly structure one or more pallets. In some embodiments, following the initial palletization (e.g., stacking two or more units of consolidated biomass) on one or more pallets, the orderly structure formed thereon may then be at least partially wrapped. For example, an external perimeter of the orderly structure of two or more units of consolidated biomass may be wrapped to prevent disassembly or dissociation of the orderly structure.

The pallets may be formed of any suitable material. In certain embodiments, one or more (and, in some cases, all) pallets comprise one or more polymers. In certain embodiments, one or more (and, in some cases, all) pallets do not comprise wood or other plant-derived components. In some cases, it may be advantageous for the pallets to be formed from one or more polymers rather than from wood or other plant-derived components to avoid introduction of non-sterilized biomass, which can serve as hosts to relevant microbial communities, into a sequestration site. In some cases, pallets formed from one or more polymers may advantageously reduce or avoid the formation of splinters, which could damage or rupture one or more layers encapsulating one or more units of consolidated biomass (e.g., briquettes and/or blocks). In certain embodiments, however, one or more (and, in some cases, all) pallets comprise wood. In some instances, one or more (and, in some cases, all) pallets may be sterilized (e.g., via UV radiation, heat, or any other sterilization method described herein) prior to being used to store and/or transport the biomass.

In some cases, palletization (e.g., stacking one or more units of consolidated biomass on one or more pallets) may advantageously allow units of consolidated biomass (e.g., briquettes and/or blocks) to be efficiently transported and/or stored. In certain cases, palletization may reduce or minimize the risk of one or more encapsulation layers (e.g., one or more layers comprising a polymeric material) being compromised during transportation and/or storage of units of consolidated biomass. FIG. 2H shows a schematic illustration of a non-limiting embodiment of palletized consolidated biomass. In FIG. 2H, a plurality of encapsulated, consolidated biomass units (e.g., blocks) 266 are stacked on pallet 268.

Much of the foregoing disclosure has focused on obtaining (e.g., receiving) unprocessed biomass and/or processing biomass. In some cases, the throughput for processing biomass is directly related to the overall rate of rate of carbon sequestration.

According to some embodiments, unprocessed biomass may be processed (e.g., comminuted, sterilized, consolidated, and/or encapsulated) at a rate of greater than or equal to 10 kg/hr., greater than or equal to 100 kg/hr., 1000 kg/hr., greater than or equal to 2000 kg/hr., greater than or equal to 3000 kg/hr., greater than or equal to 5000 kg/hr., greater than or equal to 10000 kg/hr., greater than or equal to 20000 kg/hr., greater than or equal to 50000 kg/hr., or greater than or equal to 100000 kg/hr. In some cases, the unprocessed biomass may be processed at a rate of less than or equal to 100000 kg/hr., less than or equal to 50000 kg/hr., less than or equal to 20000 kg/hr., less than or equal to 10000 kg/hr., less than or equal to 5000 kg/hr., less than or equal to 3000 kg/hr., less than or equal to 2000 kg/hr., less than or equal to 1000 kg/hr., less than or equal to 100 kg/hr., or less than or equal to 10 kg/hr. Combinations of the foregoing ranges are possible (e.g., greater than or equal to 1000 kg/hr. and less than or equal to 5000 kg/hr.). Other ranges are also possible.

In some cases, it is possible to receive processed biomass, wherein the processed biomass has been consolidated and encapsulated. In accordance with some embodiments, some unprocessed biomass and/or some processed biomass maybe received and/or processed. According to some embodiments, the received biomass is from a biomass supplier, a farm, a forest, a processor of agricultural products, and/or a processor of wood products. Other sources from which biomass may be received are also possible.

In some cases, sequestering carbon comprises storing biomass (e.g., processed biomass). Storing biomass may comprise transporting the processed biomass from a first location to a second location. For example, the first location may be a processing site and the second location may be a sequestration site, in some embodiments. In some cases, the first location may be where unprocessed biomass is obtained, received, and/or processed. According to some embodiments, the second location may comprise a sequestration site. In certain embodiments, the sequestration site may comprise a subterranean location where biomass may be stored. In some cases, the sequestration site may be a landfill. In some cases, a bottom level of the sequestration site may be located above a groundwater table (e.g., to prevent upward buoyant forces acting on a liner of the sequestration site). In certain embodiments, the sequestration site may be located above ground. As an illustrative example, the sequestration site may comprise an above-ground, lined earthen tomb or similar structure. According to some embodiments, the sequestration site may include more than one compartment and/or location in which biomass units may be stored. For example, the sequestration site may include at least two subterranean compartments where biomass may be stored. In some cases, when there are multiple compartments and/or locations, each compartment and/or location may include a liner and be configured to prevent leakage of water thereinto and/or to facilitate drainage or water therefrom.

In some embodiments, the sequestration site comprises a liner. In certain embodiments, the liner is configured to be resistant to degradation by weathering. In some cases, the liner acts as a secondary permeability barrier between the processed biomass and microbes, oxygen, and/or water vapor. In certain cases, the liner has a low rate of hydraulic conductivity (e.g., 1×10⁻⁸ m/s or less).

In some cases, the liner comprises clay and/or polyethylene. In certain embodiments, the liner is a composite liner. In some instances, the composite liner comprises one or more layers of compacted soils and one or more layers comprising a polymeric membrane (e.g., a high-density polyethylene membrane).

In some cases, the sequestration site comprises a water drainage system. In certain embodiments, the sequestration site comprises one or more drainage pipes configured to remove excess water, for example, from precipitation. In some instances, the drainage pipes may be positioned at the bottom of the sequestration site. In some cases, such drainage pipes may advantageously prevent water pools form forming in the sequestration site when portions of the site are open to the atmosphere and thus to precipitation.

In some embodiments, the sequestration site has a relatively flat floor. In certain embodiments, the floor is able to withstand compressive forces associated with units (e.g., briquettes and/or blocks) of biomass being stacked on the floor. In some cases, greater than or equal to 1000, greater than or equal to 10000, greater than or equal to 100000, greater than or equal to 1 million, greater than or equal to 10 million, greater than or equal to 100 million, greater than or equal to 1 billion, or greater than or equal to 1 trillion units of biomass may be stored on the floor of the sequestration site. In certain embodiments, the sequestration site may be configured to store greater than or equal to 100,000 tons of biomass, greater than or equal to 500,000 tons of biomass, or greater than or equal to 1 million tons of biomass per year for at least 5, 10, 20, 3, 40, 50, or 100 years.

Accordingly, in some instances, greater than or equal to 5 million tons of biomass, greater than or equal to 10 million tons of biomass, greater than or equal to 50 million tons of biomass, or greater than or equal to 100 million tons of biomass may be stored on the floor of the sequestration site.

According to some embodiments, there may be advantages associated with using a sequestration site comprising a subterranean location. For example, after storing (e.g., burying) the biomass in the subterranean location, the biomass may no longer be exposed to UV light. In some such embodiments, this may prolong the lifetime of the one or more layers encapsulating the biomass (e.g., one or more layers comprising a polymeric material). In some cases, storing (e.g., burying) the biomass in the subterranean location may provide some level of temperature control. That is, in some cases, the underground temperature may not fluctuate as much as the ambient temperature above ground due to the relatively high heat capacity of the soil as compared to the air, as well as the relatively low amounts of convection below ground relative to above ground. In some such cases, relatively stable temperatures below ground may be beneficial for minimizing biomass decomposition.

According to some embodiments, the sequestration site is sealed from an external atmosphere and/or is configured to regulate a pressure within the sequestration site. For instance, in some embodiments, an interior volume of the sequestration site may be configured to be sealed such that the biomass contained therein is not exposed to an ambient atmosphere, e.g., to maintain conditions such as a dry and/or anoxic environment to prevent decomposition. In some embodiments, the seal is a hermetic seal. Additionally, for similar reasons to the purpose of the sealing of the sequestration site, in some embodiments, the sequestration site may further include one or more desiccants contained within the interior volume of the sequestration site such that a moisture content within any air in the interior volume is low relative to an atmospheric moisture content. In some embodiments where the interior volume of the sequestration site is sealed from an ambient atmosphere, the sequestration site may further comprise a negative pressure source (e.g., a piston pump, a diaphragm pump, a peristaltic pump, or any other suitable pump for forming a vacuum) configured to withdraw gas from within the interior volume of the sequestration site to analyze the gas and monitor for any decomposition, of the biomass and/or presence of any tracer present in the biomass units.

For example, when creating a negative pressure within the interior volume of the sequestration site, a gas sample can be withdrawn for monitoring. In some such embodiments, if biomass decomposes and releases CO₂ and/or CH₄, and/or is an encapsulating layer is compromised so that and included tracer is released, a gas sample withdrawn by the vacuum device may be directed to an analysis device, e.g. comprising a sensor or other analytical device for performing gas chromatography, mass spectroscopy, etc., as described in more detail elsewhere herein) to measure a property of the gas withdrawn from the interior volume of the sequestration site. Monitoring for CO₂ and/or CH₄, in addition to or instead of in certain cases, the presence of other gases, such as O2, water vapor, tracers, etc. may be beneficial in certain cases.

It is further noted that, in some embodiments, when the sequestration site compartment(s) containing processed biomass is sealed, withdrawing gas from the interior volume of the compartment(s) may produce a vacuum therein, which may at some point reduce or eliminate the ability to withdraw additional gas samples. Accordingly, the compartment(s) may further include one or more vents configured to open to a surrounding atmosphere and/or other source of make-up gas when the interior volume of the compartment(s) is periodically sampled. This may be achieved, in some embodiments, by passively or actively opening at least one of the one or more vents to the atmosphere and/or other source of make-up gas to facilitate balancing pressures within the compartment(s) after withdrawal of gas sample(s) contained within the interior volume of the compartment(s) to prevent forming a vacuum within the compartment(s) The flow of make-up gas through the one or more vents may be passively or actively controlled, e.g., via one or more valves, such as a check valve, a solenoid valve, a piston valve, a butterfly valve, or any other valve suitable for controlling air flow. Accordingly, in some embodiments, the methods comprise monitoring the biomass, where the monitoring comprises sampling gas from the sealed compartment(s) or area(s) through an outlet to a sample collection of monitoring system and opening a vent or inlet to the sealed compartment(s) or area(a) when sampling gas through the outlet to maintain a consistent pressure within the biomass containing compartment(s).

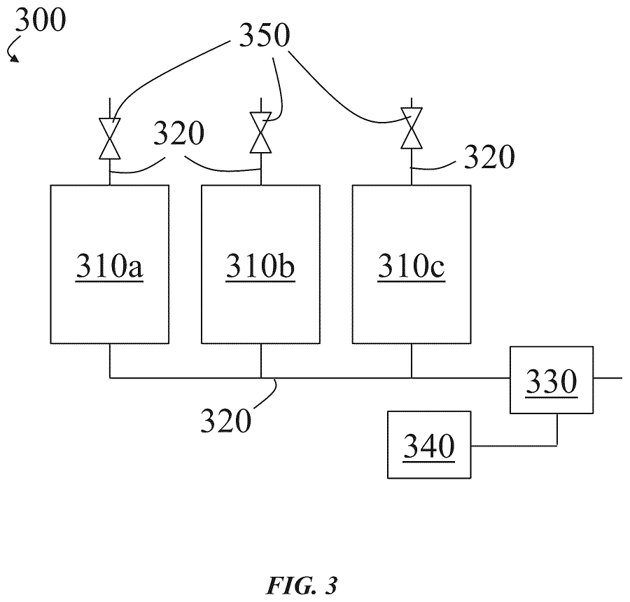

FIG. 3 shows an example gas sample collection system 300 for monitoring stored biomass within a sealed compartment(s) of a sequestration site having multiple such compartments 310a, 310b, and 310c. Each compartment 310a, 310b, and 310c is fluidically connected through outlet piping 320 to negative pressure source 330. The negative pressure source in configured to withdraw gas from the compartments 310a, 310b, and 310c and deliver the gas to an analytical device 340 comprising a gas analyzer (e.g., gas chromatography system (GC), mass spectrometer, or combination thereof (GC-MS), or other suitable analyzer) for analyzing the composition of the gas sample. Each compartment 310a, 310b, and 310c further includes a respective vent valve 350 on fluid flow lines fluidically connecting the compartments 310a, 310b, and 310c to an ambient atmosphere. The valve 350 for the corresponding compartment 310a, 310b, and 310c is configured to open to an ambient atmosphere when the system is sampling from the compartment, thereby equilibrating the pressure therein.

In accordance with some embodiments, sensors may be incorporated into and/or around the structure of the sequestration site, which may facilitate real time monitoring of biomass decomposition as described elsewhere herein.

In some cases, at least one property of the biomass (e.g., processed biomass) and/or an area where the biomass is stored may be monitored to determine the stability and/or sterility of the biomass. In some such cases, monitoring the stability may provide information about the efficiency of the carbon sequestering. The efficiency of the carbon sequestering may, in some cases, refer to the amount of carbon that remains sequestered after an amount of time (e.g., after 1 month, after 6 months, after 1 year, after 5 years, after 10 years, after 20 years, after 50 years, after 100 years, after 500 years, after 1000 years, after 1500 years, after 2000 years, after 2500 years, after 5000 years, after 10,000 years, and so forth), relative to the initial amount of sequestered carbon. Monitoring may proceed in real-time and/or occur after various time increments (e.g., every week, every 4 weeks, every year, every 5 years, every 10 years, every 20 years, every 50 years, every 100 years, every 500 years, every 1000 years, and so forth).

Monitoring may provide verification of the carbon sequestering process, which may provide more accountability for carbon sequestration projects, as well as for regulatory projects associated with carbon sequestration projects. Any of a variety of methods for monitoring the stability and/or sterility of the biomass are suitable. According to some embodiments, monitoring at least one property comprises measuring a wt. % of carbon in the biomass, a gas content (e.g., O₂, N₂, CO₂, CH₄ and/or tracer(s)) in and/or emanating from the biomass, and/or the moisture wt. % in the biomass. For instance, in some embodiments, CO₂ and/or CH₄ may emanate from the biomass, e.g., upon mechanical failure of one or more layers encapsulating the biomass and/or decomposition of the biomass. In some such embodiments, monitoring may comprise measuring a first gas content (e.g., the CO₂ and/or CH₄) present within the an interior volume of the compartment(s) of a sequestration site relative to a second gas content present in an ambient atmosphere. Such a comparison, in some embodiments, may facilitate the accurate monitoring of the amount of carbon sequestered in the biomass stored in the sequestration site. It will be appreciated that while the monitoring systems described herein may be in place to mitigate decay or decomposition, it remains desirable in the carbon sequestration processes that the biomass remains stably encapsulated so that it does not decompose.

As described elsewhere herein, the biomass may comprise an additive comprising a tracer, which is a detectable substance that is not the biomass itself or a degradation product thereof. Non-limiting examples of suitable tracers include sulfur hexafluoride, helium, hydrogen (H₂), and mercaptans. The tracer may be in the form of a solid, liquid, or gas at standard temperature and pressure (STP) and/or at the conditions prevailing during storage if not overlapping with STP. In certain embodiments, one or more tracers are selected to undergo a phase change to form one or more gaseous tracers and/or to liberate a tracer vapor. In some embodiments, the tracer is contained in a frangible container, so that upon rupture of the frangible container in the event of a mechanical collapse or other disruption of a unit(s) of biomass containing or in contact with the frangible container, the tracer is released and detected. In some embodiments, the tracer comprises a compound that be easily monitored (e.g., a gas or vapor that can be detected at a resolution of parts per billion or parts per trillion). In some embodiments, monitoring at least one property of the biomass comprises measuring a tracer content. In certain cases, the ASTM-F2391 Standard Test Method for Measuring Package and Seal Integrity Using Helium as the Tracer Gas may be used to evaluate the integrity of one or more encapsulation layers of one or more units of consolidated biomass (e.g., briquettes and/or blocks). In some embodiments, tracers are able to reduce or maintain a concentration of water in biomass, reduce or maintain a partial pressure of oxygen in or in equilibrium with the biomass, and/or reduce a live bacterial content of the biomass below that of a level of live bacteria initially contained in the biomass before exposure to the tracer. In some embodiments, the tracer(s) are incorporated within the one or more units of consolidated biomass and/or within one or more encapsulation layers coating the one or more units of consolidated biomass.

In some embodiments, the tracer may comprise an isotopically labeled compound. In accordance with some embodiments, monitoring at least one property of the biomass may comprise measuring an isotopic ratio and/or the stability of an isotope contained in the biomass. In some cases, the tracer may be useful in monitoring the decomposition of the biomass (e.g., if an isotopically labeled compound is detected, if a decomposition product of the tracer is detected) while simplifying the overall interpretation of data that may be gathered through monitoring processes.

In some embodiments, a tracer comprises two or more isotopes, such that the ratio of the two or more isotopes provides information about the biomass. In some embodiments, a ratio of two or more isotopes is at least 1:1, at least 2:1, at least 3:1, at least 5:1, at least 10:1, at least 50:1, at least 100:1, at least 1,000:1, at least 10,000:1, at least 100,000:1, at least 1,000,000:1, at least 10,000,000:1, or greater. In some embodiments, a ratio of two or more isotopes is less than or equal to 10,000,000:1, less than or equal to 1,000,000:1, less than or equal to 100,000:1, less than or equal to 10,000:1, less than or equal to 1,000:1, less than or equal to 100:1, less than or equal to 50:1, less than or equal to 10:1, less than or equal to 5:1, less than or equal to 3:1, less than or equal to 2:1, or less than or equal or 1:1. Combinations of the above-referenced ranges as also possible (e.g., at least 1:1 and less than or equal to 10,000,000:1). Other ranges are possible.

In some embodiments, one or more (and, in some cases, all) units (e.g., briquettes and/or blocks) of biomass may comprise a tracer. In certain embodiments, the tracer may be incorporated into a subset of units of biomass to provide a robust statistical sample (while maintaining high enough concentrations to enable detection in the event of a rupture or leak). In certain cases, different tracers may be placed in different locations around a sequestration site in order to provide a determination as to the location of a leak if one were to occur. In some cases, the tracer(s) may be inexpensive, stable, and/or non-reactive (chemically or biologically). In other cases, solid tracers may be reactive with water and/or oxygen to form one or more detectable gaseous tracers to enable determination of exposure of the biomass to water or oxygen, or to generation of water or oxygen by or within the biomass, e.g., as a result of decomposition or microbial activity. In some cases, the tracers may be present in low concentrations (e.g., ppm or ppb levels).

In some cases, inclusion of stable carbon isotopes directly into the biomass may provide evidence that the biomass is decomposing and forming CO₂ and/or CH₄. Isotopic ratios of CO₂ and/or CH₄ could have the unique signal of the included isotope, and it could be determined what fraction of produced CO₂ and/or CH₄ was due to decomposition of sequestered biomass. In certain cases, stable carbon isotopes may be included in the carbon polymers of the one or more encapsulation layers.

In some cases, an isotopic signature may be determined for biomass of a particular origin (e.g., a profile of naturally occurring carbon isotopes in the biomass may be determined). In some cases, monitoring at least one property of the biomass may comprise monitoring any gas emissions from a sequestration site for the presence of the isotopic signature. In some such cases, detection of the isotopic signature may indicate decomposition of biomass from the particular origin.

In some embodiments, monitoring comprises directly or indirectly measuring the mass or density of sequestered biomass. Any substantive changes in mass or density may imply that moisture ingress has occurred.

Monitoring any of the above parameters may proceed using any suitable analytical techniques known to those of ordinary skill in the art. For example, gravimetric analysis may be used to determine the mass before and/or after a set period of time. In some cases, gas chromatography may be used to measure the composition of any gas emanating from the biomass (e.g., gas that may be present due to the decomposition of the biomass). In some cases, flux towers or flux chambers may be used to measure a CO₂ flux emanating from the biomass and/or the sequestration site containing the biomass (e.g., to monitor for CO₂ that may be present due to the decomposition of the biomass). In some cases, mass spectrometry may be used to determine the amounts of different species (e.g., carbon, water, oxygen, and/or tracer(s)) in the biomass and/or the isotopic ratios present in the biomass. In some cases, sonographic techniques may be used to perform non-destructive measurement of density.

According to some embodiments, one or more sensors may be placed at one or more locations within the sequestration site. In some instances, the one or more sensors may provide real-time data. In certain cases, the one or more sensors comprise one or more load cells (force sensors), which may be used to measure mass. In certain cases, the one or more sensors comprise one or more chemical sensors configured to sense different molecules which may be indicative of biomass decomposition (e.g., CH₄, O₂, and/or isotopically labeled gases). In some cases, one or more ports may be placed at one or more locations within the sequestration site. In certain embodiments, the one or more ports may draw gases from the sequestration site, and the gases may be measured for the presence of one or more molecules (e.g., O₂, N₂, CO₂, CH₄,) and/or moisture content. In some cases, the gases may be measured for the presence of one or more volatile organic compounds (VOCs). In some cases, the presence of one or more VOCs may indicate evolution or degradation of polymers within the sequestration site.

In some cases, the carbon wt. % in the biomass may be determined in processed biomass and compared to a reference sample. In some cases, the reference sample may be the average wt. % present in a processed biomass (e.g., in a briquette and/or block). In some cases, decomposition of the biomass may be determined by measuring the change in mass and/or density of the biomass. Changes in the mass and/or density of the biomass, in some cases, may indicate that the composition of the biomass is changing. In some such cases, if the biomass is suspected of decomposing, subsequent testing may be performed to determine another property of the biomass (e.g., a carbon wt %). In some embodiments, a percent change in the mass and/or density of one or more units of biomass (e.g., briquette(s) and/or block(s)) may be less than 10%, less than 5%, less than 2%, less than 1%, less than 0.5%, or less than 0.1% over a period of at least 10 years, at least 20 years, at least 100 years, at least 200 years, at least 500 years, at least 1000 years, at least 1500 years, at least 2000 years, at least 5000 years, or at least 10,000 years.

In some embodiments, a percent change in the carbon wt. % of one or more units of biomass (e.g., briquette(s) and/or block(s)) may be less than 10%, less than 5%, less than 2%, less than 1%, less than 0.5%, or less than 0.1% over a period of at least 10 years, at least 20 years, at least 100 years, at least 200 years, at least 500 years, at least 1000 years, at least 1500 years, at least 2000 years, at least 5000 years, or at least 10,000 years.

In some embodiments, an amount of O₂, N₂, CO₂, CH₄, and/or moisture present in gases emanating from a sequestration site may be less than 5 wt. %, less than 2 wt. %, less than 1.5 wt. %, less than 1 wt. %, less than 0.5 wt. %, less than 0.1 wt. %, less than 0.05 wt. %, or less than 0.01 wt. % over a period of at least 10 years, at least 20 years, at least 100 years, at least 200 years, at least 500 years, at least 1000 years, at least 1500 years, at least 2000 years, at least 5000 years, or at least 10,000 years. In some embodiments, upon any mechanical failure of one or more layers encapsulating the one or more units of biomass, any tracer(s) and/or CO₂ and/or CH₄ may emanate from the biomass following disruption of the integrity of the encapsulating barrier layer and later decomposition of the biomass. In some such embodiments, relatively higher wt % of gases emanating from the sequestration site, compared to when the biomass units are stable, may occur.

In some cases, monitoring the biomass may further comprise reporting the amount of carbon in the biomass, for example, for confirming the efficiency of the carbon sequestration. Reporting the amount of carbon in the biomass may indicate the efficacy of the carbon sequestration scheme, and thus may be useful for ensuring the quality of the carbon sequestration technique.

Note that while the monitoring steps are described above in the context of relatively long time periods (e.g., greater than or equal to 1 year, greater than or equal to 10 years, greater than or equal to 100 years, greater than or equal to 1000 year, greater than or equal to 1500 years, greater than or equal to 2000 years, greater than or equal to 2500 years, greater than or equal to 5000 years, greater than or equal to 10,000 years), in some cases, accelerated aging experiments may be used to confirm the stability and/or sterility of the one or more layers encapsulating the biomass in a shorter period of time, which may then be used to inform the monitoring of the biomass in similar short time periods. For example, the ASTM F1980-21 test may be used to accelerate the aging of the encapsulating layer(s) (e.g., one or more layers comprising a polymeric material), wherein the decomposition of the biomass may be determined. In some cases, after aging the one or more layers in an accelerated manner, the encapsulated biomass may then be monitored in real time to determine if the biomass decomposes. Monitoring in real time in such a manner, in some cases, may proceed for greater than or equal to 1 day, greater than or equal to 1 week, greater than or equal to 4 weeks, or greater than or equal to 1 year. Testing is such a manner may be particularly informative if the one or more layers degrade during the accelerated aging experiment, as the decomposition rate of the biomass may subsequently accelerate without a fully intact encapsulation layer.

In some embodiments, if biomass decomposition is detected, one or more units of biomass (e.g., briquette(s) and/or block(s)) that are the source of the biomass decomposition may be identified and removed from the sequestration site. In some cases, the one or more units of biomass (e.g., briquette(s) and/or block(s)) may be re-encapsulated in one or more layers (e.g., one or more layers comprising a polymeric material) and re-stored in the sequestration site.

As mentioned elsewhere herein, the foregoing method steps may each be performed independently of each other, in combination, and/or in any order, whether as recited above or in a different order. In some cases, unprocessed biomass may be received and processed by comminuting the biomass, sterilizing the biomass, consolidating the biomass, encapsulating the biomass, quantifying the amount of carbon in the biomass, storing the biomass, and/or monitoring the biomass. In a preferred set of embodiments, unprocessed biomass may be obtained, comminuted such that the average particle size of the biomass is greater than or equal to 1 micron and less than or equal to 5 cm, sterilized by dehydrating the biomass, consolidated by applying a uniform pressure to the biomass to form block, log, or briquette comprising the biomass, and encapsulated using one or more layers comprising PET to form a block, log, or briquette comprising processed biomass, wherein the one or more layers comprising PET have a thickness greater than or equal to 100 nm and less than or equal to 10 mm. In some such embodiments, the block, log, or briquette comprising processed biomass may be stored by burying the block, log, or briquette in a landfill. In some embodiments, the carbon content of the briquette is monitored by using mass spectrometry and/or gas chromatography to measure the composition of the atmosphere over of the landfill (e.g., at 5 cm, 10 cm, 50 cm, 1 m, 2 m, 3 m, 5 m, or other heights above the landfill) at different times. For instance, in some such embodiments, the partial pressures of gases indicative of biomass decomposition (e.g., CO₂, CH₄) may be monitored.

In some embodiments, carbon sequestration comprises receiving biomass (unprocessed and/or processed), encapsulating the biomass, and then storing and/or monitoring the biomass. In some such cases, encapsulating the biomass in one or more layers (e.g., one or more layers comprising a polymeric material) may extend the time that the carbon may be sequestered by slowing and/or preventing the decomposition of the biomass. According to some embodiments, carbon sequestering may comprise receiving processed biomass, storing the biomass, and/or monitoring the biomass. Received processed biomass may speed the overall process of carbon sequestration.

In some embodiment, carbon sequestration may comprise receiving biomass, encapsulating the biomass, processing the biomass, storing the biomass, and monitoring the biomass. In some such cases, encapsulating the biomass may occur before other processing steps, such as consolidating and/or sterilizing. In some cases, biomass may be encapsulated in one or more layers (e.g., one or more layers comprising a polymeric material) that are deposited (e.g., via wrapping, shrink wrapping, spraying, brushing, and/or dip-coating) on the biomass. In some cases, biomass may be encapsulated in pre-engineered bags, wherein the biomass may be processed within the pre-engineered bags.

The biomass processing and/or storage described herein may be useful for many applications. For example, processing and storing the biomass such that the processed biomass resists decomposition for a period of time (e.g., at least 1 year, 5 years, 10 years, 50 years, 100 years, 500 years, 1000 years, 1500 years, 2000 years, 2500 years, 5000 years, 10,000 years, or more) can be used to capture and store carbon and prevent it from reentering the atmosphere. In such a manner, the processing and/or storing of biomass may change the atmospheric composition (e.g., over long time periods) and potentially provide beneficial changes to the environment.

In some embodiments wherein biomass is consolidated into briquettes and/or blocks, logs, pellets, etc. and encapsulated, depending on the composition of the encapsulating layer(s) (e.g., the polymeric material) and/or the structural integrity of the consolidated biomass, the briquettes and/or blocks may be useful as building materials. For example, the briquettes and/or blocks may function similarly to clay bricks in architecture, depending on the resistance of the briquettes and/or blocks to degradation due to weathering (e.g., UV radiation exposure, precipitation, etc.). In some cases, using briquettes and/or blocks comprising biomass may reduce the carbon footprint of building new structures by simultaneously sequestering carbon and/or avoiding traditional building materials.

Sequestering carbon in the manner described herein may be advantageous to conform with regulatory policy. For example, while some companies may perform operations that emit carbon into the atmosphere, participation in carbon sequestering (e.g., via the process described herein) may “offset” or “reduce” their emissions and may even result in a net negative carbon emission. By using the improved methods for processing and/or monitoring biomass for carbon sequestration described in the present disclosure, more accurate and accountable carbon sequestration may be utilized. Some aspects of the present disclosure that may be particularly relevant to this end may be the monitoring of the biomass, which may ensure that carbon is sequestered from the atmosphere for relatively long times. In some cases, conforming to regulatory policy may motivate companies to invest in methods for reducing their carbon footprint to avoid potentially steep fines from regulatory agencies. Accordingly, in addition to regulatory policies, there may also be financial incentive for companies to adopt the carbon sequestration techniques described herein.

The following examples are intended to illustrate certain embodiments of the present invention, but does not exemplify the full scope of the invention.

### PROPHETIC EXAMPLE 1

This example describes a process of carbon sequestration, according to some embodiments.

Biomass in the form of sawdust, wood waste, rice hulls, rice straw, wheat straw, and sugar cane bagasse is obtained. The biomass is conveyed and fed into a hammer mill, where the biomass is comminuted to reduce and uniformize the average particle size of the biomass. The comminuted biomass is comminuted to a rotary drum heater where the biomass is dehydrated at 170° C. for 20 minutes to sterilize. The moisture content within the biomass is reduced within the rotary drum heater to no more than 12 wt. % of the biomass. Following dehydration, the biomass is conveyed to a briquetting machine, where the comminuted biomass is consolidated (i.e., densified) into briquettes. Note that, periodically, the biomass is sampled and a carbon content is determined using an elemental analyzer.

The briquettes are then conveyed to an encapsulation machine, a HarpakUlma FM300 machine, where the briquettes are encapsulated and hermetically sealed within a durable composite barrier film. In the encapsulation machine, a film is positioned over the around each briquette, whereafter the film is sealed forming seams surrounding the briquette using a heating jaw. Here, each briquette is individually encapsulated. The barrier film is a three-layer film that includes a polyamide outer layer, a metallized polyethylene terephthalate middle layer, and an inner layer that is a coextruded material comprising polyethylene and polyamide, where the inner layer seals the barrier film when the encapsulation machine applies the heat jaw.

Each briquette is then conveyed from the encapsulation machine. A check weigher measures the weight of each individual briquette, whereafter the amount of carbon contained within each briquette is determined using the weight and the carbon content determined earlier using the elemental analyzer.

The encapsulated briquettes are further conveyed to a location where they are palletized. The pallet containing the individually encapsulated briquettes is then wrapped to ease transport of the pallet of briquettes.

The biomass is then transported from the processing site to the sequestration site. The sequestration site includes multiple storage cells, each storage cell being excavated to a depth of 12 feet and incorporates a clay liner and geomembrane liners to prevent water infiltration therein. The sequestration site is engineered with a rainwater collection system to prevent any water pooling within the interior volume of the storage cells of the sequestration site where the biomass briquettes are stored. The rainwater collection system additionally facilitates testing of the runoff water for any environmental contaminants, such as microplastics, which may result from degradation of the encapsulation material (i.e., the barrier films described above). Each storage cell is filled with biomass briquettes and then capped with 2 feet of clay and 18 inches of soil.

Each of the separate storage cells within the sequestration site is monitored for the generation of CO₂ or CH₄ by sampling the head space within each storage cell after closing (i.e., capping with the clay and soil) using a Picarro G220-i Analyzer. Samples obtained from the head space of each storage cell is compared with a concurrently collected background sample to determine any increase in the concentration of CO₂, CH₄, and/or δ13C, and thus any degradation of the barrier films of the briquettes and/or decomposition of biomass contained therein. This sampling is performed quantitatively such that the level and rate of decomposition is determined.

### PROPHETIC EXAMPLE 2

This example describes a process of carbon sequestration, according to some embodiments.

In this case, biomass is received, comminuted, and dehydrated as in Example 1. Following comminution, the comminuted biomass is conveyed to an extrusion line (rather than a briquetting machine as in Example 1), where the comminuted biomass is consolidated (i.e., densified) and extruded into cylindrical biomass units. The cylindrical biomass units are then similarly encapsulated, transported, stored, and monitored as in Example 1.

### EXAMPLE 3

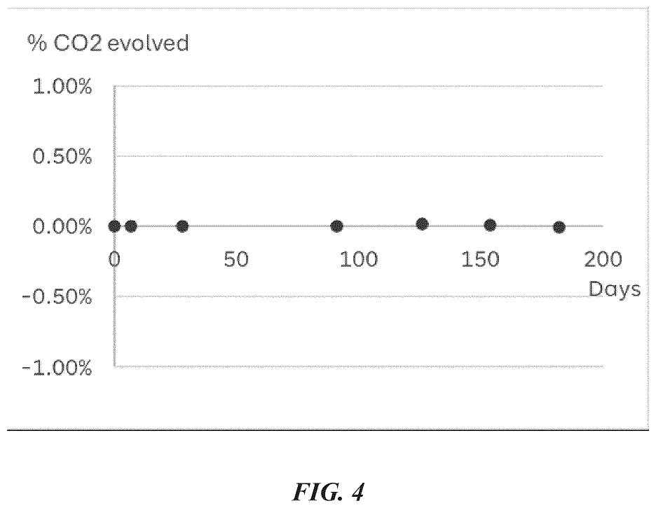

In this experiment, the rotary drum drying technique as would be used in Prophetic Example 1 was tested to assure sufficient sterility of the dehydrated biomass was achieved. Consolidated biomass blocks were produced and dehydrated using a rotary drum heater at 170° C. for 20 minutes resulting in a final moisture content measured to be between 9.2 and 9.8 wt. %. To test for microbial activity, the dehydrated blocks were chopped into 3 inch×3 inch pieces that could fit within a glass chamber. Pellets of potassium hydroxide (KOH) were placed into the glass chamber with the pieces, and the glass chamber was closed and hermetically sealed for select periods of time. Data points were taken by opening the chamber, taking out the pellets and titrating them using 0.5 N hydrochloric acid (HCl) solution to determine the volume of CO₂ that was generated during the duration of the experiment. These results, shown in FIG. 4, demonstrate that no detectable CO₂ was generated over the first 6 months of experimental testing. This indicates that the dehydration process has effectively halted biomass decomposition.

### EXAMPLE 4

Utilizing biomass for carbon removal requires that the carbon within the biomass is prevented from decomposing and being released as carbon dioxide or methane. This is currently done through processing (i.e., drying) the biomass and storing it in conditions that prevent decay (i.e., in dry or anoxic conditions). However, if it were possible to process the biomass itself such that it was inherently recalcitrant to decomposition, that would reduce the need for protection during storage. In that case, if water were to get into the storage chamber, the biomass itself would repel it and therefore prevent decomposition from occurring.

This disclosure is to create a hydrophobic composite material that is recalcitrant to decomposition through the simultaneous densification of biomass with a polymer stream. Ideally this would be done with waste polymer that would otherwise need disposal. For example, coextruding polymer with biomass into a pellet creates a composite pellet that can be water-repellant. This water-repellant behavior can make the composite pellet recalcitrant to decomposition, an ideal set of characteristics for carbon removal. In embodiments, provided herein is biomass, which is coextruded with a polymer, which is hydrophobic. In embodiments, biomass, which is coextruded with a polymer, which is hydrophobic, is not wrapped in an encapsulating layer. In embodiments, biomass, which is coextruded with a polymer, which is hydrophobic, is sequestered. In embodiments, the biomass, which is coextruded with a polymer, which is hydrophobic, is stored in a storage facility. In embodiments, the storage facility is a landfill.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teaching of the present invention is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for sequestering carbon, the method comprising:
receiving biomass; and
processing the biomass for sequestration, wherein processing comprises:
comminuting the biomass;
sterilizing the biomass;
consolidating the biomass to form a plurality of consolidated biomass units;
encapsulating the consolidated biomass units; and
quantifying a carbon content of the biomass.

2. The method of claim 1, wherein the sterilizing step occurs after the comminuting step and before the consolidating step.

3. The method of claim 1, wherein the sterilizing step occurs after the consolidating step and before the encapsulating step.

4. (canceled)

5. (canceled)

6. (canceled)

7. A method for sequestering carbon, the method comprising:
receiving biomass; and
processing the biomass for sequestration, wherein processing comprises:
consolidating the biomass to form a plurality of consolidated biomass units;
encapsulating the consolidated biomass units sufficiently to prevent microbial activity and decay of encapsulated biomass for at least 100 years when the biomass is stored in darkness under standard atmospheric conditions; and
quantifying a carbon content of the biomass.

8. The method of claim 7, wherein in the encapsulating step the consolidated biomass units are encapsulated sufficiently to prevent microbial activity and decay of encapsulated biomass for at least 100 years when the biomass is stored in conditions where exposed to visible light.

9. A method for sequestering carbon, the method comprising:
receiving processed biomass, wherein the processed biomass has been consolidated and encapsulated; and
monitoring at least one property of the processed biomass or an area where the processed biomass is stored to determine the stability and/or sterility of the processed biomass.

10. The method of claim 9, wherein the monitoring comprises sampling gas from a hermetically sealed area in which the processed biomass is stored through a first outlet; and
opening a vent to the hermetically sealed area when sampling gas through the first inlet to maintain a pressure within the hermetically sealed area.

11. (canceled)

12. (canceled)

13. (canceled)

14. The method of claim 1, wherein the biomass is solid biomass.

15. The method claim 1, wherein the biomass is plant-derived biomass.

16. The method of claim 1, wherein the biomass comprises waste from agricultural harvesting and/or processing, waste from lumber harvesting and/or processing, and/or grasses.

17. The method of claim 1, wherein the biomass comprises palm oil waste, sugarcane bagasse, rice husks, soybean hulls, coconut shell husks, rice straw, wheat straw, corn stover, logs, lumber residue, bark, sawdust, wood chips, boles, branches, miscanthus, switchgrass, and/or seaweed.

18. The method of claim 1, wherein the received biomass is from one or more farms, forests, agricultural processing facilities, lumber processing facilities, forestry companies, municipal governments, grocery stores, restaurants, and/or food processing facilities.

19. The method claim 1, wherein comminuting the biomass comprises grinding, shredding, pounding, chopping, milling, and/or cutting the biomass from articles having a first average largest dimension to particles having a second average largest dimension less than the first average largest dimension.

20. The method of claim 19, wherein the first average largest dimension is greater than or equal to 5 cm and/or less than or equal to 25 m.

21.-109. (canceled)
